(19) United States
(12) Patent Application Publication
(10) Pub. No.: US 2010/0240713 A1
(43) Pub. Date: Sep. 23, 2010

(54) AROMATIC AND HETEROAROMATIC COMPOUNDS USEFUL IN TREATING IRON DISORDERS

(75) Inventors: Jean-Jacques Cadieux, Burnaby (CA); Mikhail Chafeev, Burnaby (CA); Julia Fonarev, Richmond (CA); Jianmin Fu, Coquitlam (CA); Rajender Kamboj, Burnaby (CA); Vishnumurthy Kodumuru, Burnaby (CA); Serguei Sviridov, Burnaby (CA); Zaihui Zhang, Vancouver (CA)

Correspondence Address: Seed IP Law Group PLLC, 701 Fifth Avenue, Suite 5400, Seattle, WA, 98104, US

(73) Assignee: XENON PHARMACEUTICALS INC., Burnaby, BC (CA)

(21) Appl. No.: 12/663,104

(22) PCT Filed: Jun. 5, 2008
(86) PCT No.: PCT/US2008/065949

§ 371 Date: May 24, 2010

Related U.S. Application Data
(60) Provisional application No. 60/942,167, filed on Jun. 5, 2007.

(51) Int. Cl. A61K 31/44; C07C 211/50; C07C 261/04; C07C 22/04; C07C 33/26; C07D 333/12; C07D 333/16; C07D 495/02; C07D 249/14; C07D 213/53; A61K 31/135; A61K 31/165; A61K 31/03; A61K 31/047; A61K 31/381; A61K 31/4196; A61P 43/00

(52) U.S. Cl. 514/357; 564/305; 564/105; 570/185; 568/811; 549/29; 549/80; 549/78; 549/50; 548/265.6; 546/331; 514/646; 514/609; 514/751; 514/738; 514/438; 514/443; 514/383

(57) ABSTRACT

This invention is directed to compounds of formula (I), wherein m, formula (II), R¹, R² and R³ are as defined herein, as a stereoisomer, enantiomer, tautomer thereof or mixtures thereof; or a pharmaceutically acceptable salt, solvate or prodrug thereof, for the treatment of iron disorders. This invention is also directed to pharmaceutical compositions comprising the compounds and methods of using the compounds to treat iron disorders.

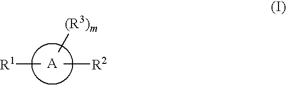

### FIELD OF THE INVENTION

The present invention is directed to aromatic and heteroaromatic compounds which are divalent metal transporter-1 inhibitors. The compounds of the invention, and pharmaceutical compositions comprising the compounds, are therefore useful in treating iron disorders.

### BACKGROUND OF THE INVENTION

Iron is an essential metal for life because it is a key constituent of a family of fundamental proteins, which includes hemoglobin, cytochromes, and NADH-coenzyme Q reductase. Maintaining body iron homeostasis is paramount to health because iron deficiency or excess results in morbidity and mortality.

Divalent metal transporter-1 (DMT1), also known as natural resistance-associated macrophage protein-2 (NRAMP2) and divalent cation transporter-1 (DCT1), is a ubiquitiously expressed transmembrane protein involved in the maintenance of iron levels in the body. DMT1 is particularly important for iron absorption in the duodenum of the small intestine, where it is localized in the cytoplasm and brush border membrane of the villus enterocytes and mediates the influx of dietary non-heme iron from the intestinal lumen into the enterocytes (Gunshin et al., J. Clin. Invest., 2005, 115:1258-1266). Once dietary iron is absorbed across the intestinal wall, there is no physiologic mechanism for excreting iron from the body. Thus, excess absorbed iron is largely retained in the body and can accumulate throughout life. Excess accumulation of iron leads to considerable tissue damage and increased subsequent disease risk such as, for example, cirrhosis or hepatocellular carcinoma. Therefore, DMT1 is the primary focal point of controlling intestinal iron absorption for the maintenance of body iron homeostatsis.

There is compelling evidence to support that DMT1 activity is tightly associated with many common diseases, such as, but not limited to, primary iron overload disorders, especially diseases related to hereditary hemochromatosis (Rolfs et al., Am. J. Physiol. Gastrointest. Liver Physiol., 2002, 282(4):G598-607). Further, DMT1 plays a significant role in intestinal iron hyperabsorption in patients suffering from hypochromic microcytic anemias and related disorders (Morgan et al., Blood Cell, Molecules, and Diseases, 2002, 29(3):384-399).

To date, there are only three known small-molecule, drug-like compounds that specifically modulate or inhibit DMT1 (Welti et al., Chem. Biol., 2006, 13:965-972). Accordingly, there is an unmet medical need to treat iron disorders, preferably primary iron overload and transfusional iron overload, including thalassemia, in mammals, preferably in humans, effectively and without adverse side effects. The present invention provides compounds and methods to meet these critical needs.

### SUMMARY OF THE INVENTION

The present invention is directed to aromatic and heteroaromatic compounds of the invention and pharmaceutical compositions comprising the compounds for the treatment of iron disorders.

Accordingly, in one aspect this invention provides compounds of formula (I):

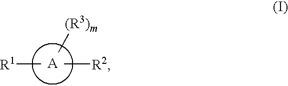

wherein:
m is 0, 1, 2, 3, or 4;

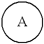

is aryl or heteroaryl;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R³ is independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁹ is independently hydrogen or alkyl; and
each R⁹ is alkyl;
as a stereoisomer, enantiomer, tautomer thereof or mixtures thereof;
or a pharmaceutically acceptable salt, solvate or prodrug thereof.

In another aspect, the invention provides pharmaceutical compositions comprising a pharmaceutically acceptable excipient and a compound of formula (I), as a stereoisomer, enantiomer, tautomer thereof or mixtures thereof, or as a pharmaceutically acceptable salt, solvate or prodrug thereof.

In another aspect, the invention provides methods for treating an iron disorder in a mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating a disease or condition associated with an iron disorder in a mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating a disease or condition associated with an iron disorder in a mammal due to accumulation of iron in the body tissues of the mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating an iron disorder in a mammal or a disease or condition associated with an iron disorder in a mammal, wherein the iron disorder, disease or condition is associated with increased DMT1 activity and wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of inhibiting the activity of DMT1 in a cell, preferably a mammalian cell, wherein the methods comprise contacting the mammalian cell with a DMT1-inhibitory amount of a compound of the invention, as set forth above, as a stereoisomer, enantiomer, tautomer thereof or mixtures thereof, or a pharmaceutically acceptable salt, solvate or prodrug thereof.

In another aspect, the invention provides methods of treating an iron disorder in a mammal, wherein the iron disorder is ameliorated by the inhibition of the activity of DMT1 in the mammal and wherein the methods comprise administering to the mammal a DMT1-inhibiting amount of a compound of the invention, as set forth above, as a stereoisomer, enantiomer, tautomer thereof or mixtures thereof, or a pharmaceutically acceptable salt, solvate or prodrug thereof, or a DMT1-inhibiting amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides pharmaceutical therapy in combination with one or more other compounds of the invention or one or more other accepted therapies or as any combination thereof to increase the potency of an existing or future drug therapy or to decrease the adverse events associated with the accepted therapy.

In one embodiment, the invention relates to a pharmaceutical composition combining compounds of the present invention with established or future therapies for the indications listed in the invention.

In another aspect, this invention is directed to the use of the compounds of the invention, as set forth above, as a stereoisomer, enantiomer, tautomer thereof or mixtures thereof, or a pharmaceutically acceptable salt, solvate or prodrug thereof, or the use of a pharmaceutical composition comprising a pharmaceutically acceptable excipient and a compound of the invention, as set forth above, as a stereoisomer, enantiomer, tautomer thereof or mixtures thereof, or a pharmaceutically acceptable salt, solvate or prodrug thereof, in the preparation of a medicament for the treatment of iron disorders in a mammal.

### DETAILED DESCRIPTION OF THE INVENTION

### Definitions

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Amino” refers to the —NH₂ radical.

“Cyano” refers to the —CN radical.

“Hydroxy” refers to the —OH radical.

“Imino” refers to the ═NH substituent.

“Nitro” refers to the —NO₂ radical.

“Oxo” refers to the ═O substituent.

“Thioxo” refers to the ═S substituent.

“Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₚR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkynyl” refers to a straight or branched hydrocarbon chain radical group comprising solely of carbon and hydrogen atoms, containing at least one triple bond, optionally containing at least one double bond, having from two to twelve carbon atoms, preferably two to eight carbon atoms and which is attached to the rest of the molecule by a single bond, for example, ethynyl, propynyl, butynyl, pentynyl, hexynyl, and the like. Unless stated otherwise specifically in the specification, an alkynyl group may be optionally substituted by one or more of the following substituents: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkynylene” or “alkynylene chain” refers to a straight or branched divalent hydrocarbon chain linking the rest of the molecule to a radical group, consisting solely of carbon and hydrogen, containing at least one triple bond and having from two to twelve carbon atoms, e.g., propynylene, n-butynylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain. Unless stated otherwise specifically in the specification, an alkynylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkoxy” refers to a radical of the formula —ORₐ where Rₐ is an alkyl radical as defined above containing one to twelve carbon atoms. The alkyl part of the alkoxy radical may be optionally substituted as defined above for an alkyl radical.

“Alkoxyalkyl” refers to a radical of the formula —R_{b}—O—Rₐ where R_{b} is an alkylene chain as defined above and Rₐ is an alkyl radical as defined above. The oxygen atom may be bonded to any carbon in the alkylene chain and in the alkyl radical. The alkyl part of the alkoxyalkyl radical may be optionally substituted as defined above for an alkyl group. The alkylene chain part of the alkoxyalkyl radical may be optionally substituted as defined above for an alkylene chain.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may included fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals optionally substituted by one or more substituents independently selected from the group consisting of alkyl, akenyl, halo, haloalkyl, haloalkenyl, cyano, nitro, aryl, aralkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁸, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Aralkyl” refers to a radical of the formula —R_{b}—R_{C} where R_{b} is an alkylene chain as defined above and Rₑ is one or more aryl radicals as defined above, for example, benzyl, diphenylmethyl and the like. The alkylene chain part of the aralkyl radical may be optionally substituted as described above for an alkylene chain. The aryl part of the aralkyl radical may be optionally substituted as described above for an aryl group.

“Aralkenyl” refers to a radical of the formula —R_{d}—R_{c} where R_{d} is an alkenylene chain as defined above and R_{c} is one or more aryl radicals as defined above. The aryl part of the aralkenyl radical may be optionally substituted as described above for an aryl group. The alkenylene chain part of the aralkenyl radical may be optionally substituted as defined above for an alkenylene group.

“Aralkynyl” refers to a radical of the formula —RₑR_{c} where Rₑ is an alkynylene chain as defined above and R_{c} is one or more aryl radicals as defined above. The aryl part of the aralkynyl radical may be optionally substituted as described above for an aryl group. The alkynylene chain part of the aralkynyl radical may be optionally substituted as defined above for an alkynylene chain.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptly, and cyclooctyl. Polycyclic radicals include, for example, adamantyl, norbornyl, decalinyl, bicyclo[2.2.1]heptanyl, and the like. Unless otherwise stated specifically in the specification, the term “cycloalkyl” is meant to include cycloalkyl radicals which are optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Cycloalkylalkyl” refers to a radical of the formula —R_{b}R_{g} where R_{b} is an alkylene chain as defined above and R_{g} is a cycloalkyl radical as defined above. The alkylene chain and the cycloalkyl radical may be optionally substituted as defined above.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkenyl” refers to an alkenyl radical, as defined above, that is substituted by one or more halo radicals, as defined above. The alkenyl part of the haloalkyl radical may be optionally substituted as defined above for an alkenyl group.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiomorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkyene chain. The heterocyclyl part of the heterocyclylalkyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, the term “heteroaryl” is meant to include heteroaryl radicals as defined above which are optionally substituted by one or more substituents selected from the group consisting of alkyl, alkenyl, alkoxy, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl.

“Heteroarylalkyl” refers to a radical of the formula —R_{b}Rᵢ where R_{b} is an alkylene chain as defined above and Rᵢ is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of the invention being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the binding affinity to pharmacologically important site of action on DMT1. Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of the invention can generally be prepared by conventional techniques known to those skilled in the art or by processes analogous to those described in the Preparations and Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reducation, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its coversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g. cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. When a functional group is described as “optionally substituted,” and in turn, substitutents on the functional group are also “optionally substituted” and so on, for the purposes of this invention, such iterations are limited to five, preferably such iterations are limited to two.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts. The term also includes quaternary ammonium salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Therapeutically effective amount” refers to that amount of a compound of the invention which, when administered to a mammal, preferably a human, is sufficient to effect treatment, as defined below, of an iron disorder or a disease or condition associated with an iron disorder, in the mammal, preferably a human. The amount of a compound of the invention which constitutes a “therapeutically effective amount” will vary depending on the compound, the iron disorder, disease or condition and its severity, the manner of administration, and the age of the mammal to be treated, but can be determined routinely by one of ordinary skill in the art having regard to his own knowledge and to this disclosure. Preferably, for purposes of this invention, a “therapeutically effective amount” is that amount of a compound of invention which is sufficient to inhibit the activity of DMT1.

“Treating” or “treatment”, as used herein, covers the treatment of an iron disorder in a mammal, preferably a human, or a disease or condition associated with an iron disorder in a mammal, preferably a human, and includes:

(i) preventing an iron disorder in a mammal, or a disease or condition associated with an iron disorder in the mammal, from occurring in the mammal;

(ii) inhibiting an iron disorder in a mammal, or a disease or condition associated with an iron disorder in the mammal, i.e., arresting its development;

(iii) relieving an iron disorder in a mammal, or a disease or condition associated with an iron disorder in the mammal, i.e., causing regression of the iron disorder or the disease or condition;

(iv) relieving the symptoms of an iron disorder in a mammal, or a disease or condition associated with an iron disorder in the mammal, i.e., relieving the symptoms without addressing the underlying iron disorder, disease or condition; or

(v) restoring and/or maintaining normal serum iron levels, transferrin saturation, serum ferritin, liver iron and/or bodily iron levels in a mammal having an iron disorder or having a disease or condition associated with an iron disorder.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any compounds of the invention.

Also within the scope of the invention are intermediate compounds of the compounds of the invention (i.e., compound which are used and/or formed in the preparation of the compounds of the invention) and all polymorphs of the aforementioned species and crystal habits thereof.

The chemical naming protocol and structure diagrams used herein are a modified form of the I.U.P.A.C. nomenclature system, using the ChemDraw Versions 10.0 or 11.0 software naming program (CambridgeSoft), wherein the compounds of the invention are named herein as derivatives of the central core structure, e.g., the aryl or heteroaryl central structure. For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency.

Thus, for example, a compound of formula (I) wherein m is 2, each R³ is ethoxycarbonyl,

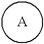

is phenyl, and R¹ and R² are the same and are each —CH₂—S—C(═NH)NH₂; e.g., a compound of the following formula:

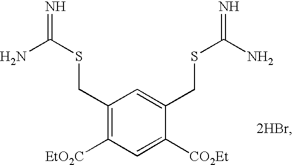

is named herein as diethyl 4,6-bis(carbamimidoylthiomethyl)isophthalate.

### EMBODIMENTS OF THE INVENTION

Of the various aspects of the invention set forth above in the Summary of the Invention, certain embodiments are preferred.

Of the compounds of formula (I) described above in the Summary of the Invention, one embodiment is wherein the compound of formula (I) is a compound of formula (Ia):

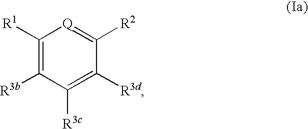

wherein:
Q is —C(R^{3a})═ or —N═;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ia) is a compound of formula (Ia) wherein:
Q is —C(R^{3a})═;
R¹ and R² are the same and are selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Another embodiment of the compounds of formula (Ia) is a compound of formula (Ia) wherein:
Q is —C(R^{3a})═;
R¹ and R² are each —R⁶—S—C(═NR⁴)N(R⁴)R⁵;
R^{3a}, R^{3b}, R^{3c} and R^{3d} are each independently selected from the group consisting of hydrogen, alkyl, halo, —R⁶—OR⁷, —R⁶—CN, —R⁶—C(O)OR⁸ and —R⁶—S—C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Ia) is the compound of formula (Ia) selected from the group consisting of:
1,3-phenylenebis(methylene)dicarbamimidothioate;
(2,4,6-trimethyl-1,3-phenylene)bis(methylene)dicarbamimidothioate;
(2-fluoro-1,3-phenylene)bis(methylene)dicarbamimidothioate;
1,3-phenylene dicarbamimidothioate;
(5-methyl-1,3-phenylene)bis(methylene)dicarbamimidothioate;
(2,4,6-trimethylbenzene-1,3,5-triyl)tris(methylene)tricarbamimidothioate;
2-{1-[3-(1-carbamimidoylsulfanyl-1-methylethyl)phenyl]-1-methylethyl}isothiourea;
(2-cyano-1,3-phenylene)bis(methylene)dicarbamimidothioate;
(4,6-dimethyl-1,3-phenylene)bis(methylene)dicarbamimidothioate; diethyl 4,6-bis(carbamimidoylthiomethyl)isophthalate;
(5-bromo-4,6-dimethyl-1,3-phenylene)bis(methylene)dicarbamimidothioate;
(2,4,5,6-tetramethyl-1,3-phenylene)bis(methylene)dicarbamimidothioate;
2-{1-[3-(1-carbamimidoylsulfanylethyl)-2,4,6-trimethylphenyl]ethyl}isothiourea;
(2-hydroxy-5-methyl-1,3-phenylene)bis(methylene)dicarbamimidothioate;
1,3-di[(methylamidino)thiomethyl]-2,4,6-trimethylbenzene;
(5-hydroxy-2,4,6-trimethyl-1,3-phenylene)bis(methylene)dicarbamimidothioate;
(2,4,5,6-tetrachloro-1,3-phenylene)bis(methylene)dicarbamimidothioate;
(2-methoxy-5-methyl-1,3-phenylene)bis(methylene)dicarbamimidothioate;
(2-methyl-1,3-phenylene)bis(methylene)dicarbamimidothioate;
(4-methoxy-1,3-phenylene)bis(methylene)dicarbamimidothioate;
(5-methoxy-1,3-phenylene)bis(methylene)dicarbamimidothioate;
(4,6-dibromo-1,3-phenylene)bis(methylene)dicarbamimidothioate; and
(4,6-diisopropyl-1,3-phenylene)bis(methylene)dicarbamimidothioate.

Another embodiment of the compounds of formula (Ia) is a compound of formula (Ia) wherein:
Q is —C(R^{3a})═;
R¹ and R² are the same and selected from —R⁶—N(R⁷)C(═NCN)N(R⁴)R⁵ and —R⁶—C(═NR⁴)N(R⁴)R⁵;
R^{3a}, R^{3b}, R^{3c} and R^{3d} are each independently selected from the group consisting of hydrogen, alkyl, halo, —R⁶—OR⁷, —R⁶—CN, —R⁶—C(O)OR⁸ and —R⁶—S—C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Ia) is the compound of formula (Ia) selected from the group consisting of:
1,3-di[(2-cyano-3-methylguanidino)methyl]-2,4,6-trimethylbenzene; and
2,2′-(1,3-phenylene)diacetimidamide.

Another embodiment of the compounds of formula (Ia) is a compound of formula (Ia) wherein:
Q is —C(R^{3a})═;
R¹ and R² are each —R⁶—N(R⁷)C(═NR⁴)N(R⁴)R⁵;
R^{3a}, R^{3b}, R^{3c} and R^{3d} are each independently selected from the group consisting of hydrogen, alkyl, halo, —R⁶—OR⁷, —R⁶—CN, —R⁶—C(O)OR⁸ and —R⁶—S—C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Ia) is the compound of formula (Ia) that is N-(3-guanidinomethyl-2,4,6-trimethylbenzyl)guanidine.

Another embodiment of the compounds of formula (Ia) is a compound of formula (Ia) wherein:
Q is —N═;
R¹ and R² are the same and are selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Another embodiment of the compounds of formula (Ia) is a compound of formula (Ia) wherein:
Q is —N═;
R¹ and R² are each —R⁶—S—C(═NR⁴)N(R⁴)R⁵;
R^{3b}, R^{3c} and R^{3d} are each independently selected from the group consisting of hydrogen, alkyl, and halo;
each R⁴ and R⁵ is independently hydrogen, alkyl, or —OR⁷;
each R⁶ is independently a direct bond or a straight or branched alkylene chain; and
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl.

Another embodiment of the compounds of formula (Ia) is the compound of formula (Ia) selected from the group consisting of:
pyridine-2,6-diylbis(methylene)dicarbamimidothioate; and
(2,4,6-trimethylpyridine-3,5-diyl)bis(methylene)dicarbamimidothioate.

Of the compounds of formula (I) described above in the Summary of the Invention, one embodiment is wherein the compound of formula (I) is a compound of formula (Ib):

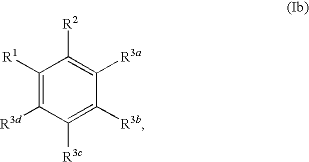

wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ib) is a compound of formula (Ib) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Another embodiment of the compounds of formula (Ib) is a compound of formula (Ib) wherein:
R¹ and R² are the same and are —R⁶—S—C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Ib) is the compound of formula (Ib) selected from the group consisting of:
(1,2-phenylene)bis(methylene)dicarbamimidothioate; and
(3,4,5,6-tetramethyl-1,2-phenylene)bis(methylene)dicarbamimidothioate.

Of the compounds of formula (I) described above in the Summary of the Invention, one embodiment is wherein the compound of formula (I) is a compound of formula (Ic):

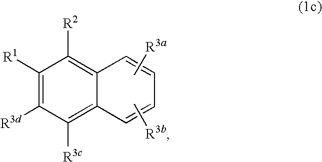

wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ic) is a compound of formula (Ic) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Another embodiment of the compounds of formula (Ic) is a compound of formula (Ic) wherein:
R¹ and R² are both —R⁶—S—C(═NR⁴)N(R⁴)R⁶;
R^{3a}, R^{3b}, R^{3c} and R^{3d} are each independently selected from the group consisting of hydrogen, alkyl, halo, haloalkyl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Ic) is the compound of formula (Ic) that is naphthalene-1,2-diylbis(methylene)dicarbamimidothioate.

Of the compounds of formula (I) described above in the Summary of the Invention, one embodiment is wherein the compound of formula (I) is a compound of formula (Id):

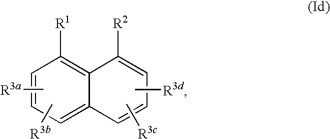

wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;

R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Id) is a compound of formula (Id) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Another embodiment of the compounds of formula (Id) is a compound of formula (Id) wherein:
R¹ and R² are both —R⁶—S—C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Id) is the compound of formula (Id) that is naphthalene-1,8-diylbis(methylene)dicarbamimidothioate.

Of the compounds of formula (I) described above in the Summary of the Invention, one embodiment is wherein the compound of formula (I) is a compound of formula (Ie):

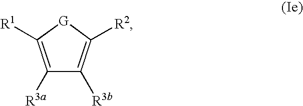

wherein:
G is —O— or —S—;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (Ie) is a compound of formula (Ie) wherein:
G is —O— or —S—;
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Another embodiment of the compounds of formula (Ie) is a compound of formula (Ie) wherein:
G is —S—;
R¹ and R² are the same and selected from —R⁶—S—C(═NR⁴)N(R⁴)R⁵ and —R⁶—C(O)—S—C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Ie) is the compound of formula (Ie) selected from the group consisting of:
2-(5-carbamimidoylsulfanecarbonyl-3,4-dichlorothiophene-2-carbonyl)isothiourea;
thiophene-2,5-diylbis(methylene)dicarbamimidothioate;
(3,4-diphenylthiophene-2,5-diyl)bis(methylene)dicarbamimidothioate; and
(3,4-dimethylthiophene-2,5-diyl)bis(methylene)dicarbamimidothioate.

Of the compounds of formula (I) described above in the Summary of the Invention, one embodiment is wherein the compound of formula (I) is a compound of formula (If):

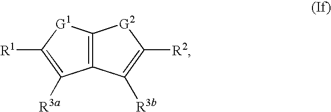

wherein:
G¹ and G² are both —O—;
or G¹ and G² are both —S—;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

One embodiment of the compounds of formula (If) is a compound of formula (If) wherein:
G¹ and G² are both —O—;
or G¹ and G² are both —S—;
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N (R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

Another embodiment of the compounds of formula (If) is a compound of formula (If) wherein:
G¹ and G² are both —S—;
R¹ and R² are the same and selected from —R⁶—S—C(═NR⁴)N(R⁴)R⁵ and —R⁶—C(O)—S—C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (If) is the compound of formula (If) that is (3,4-dimethylthieno[2,3-b]thiophene-2,5-diyl)bis(methylene) dicarbamimidothioate.

Of the compounds of formula (I) described above in the Summary of the Invention, one embodiment is wherein the compound of formula (I) is a compound of formula (Ig):

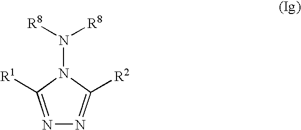

wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

One embodiment of the compounds of formula (Ig) is a compound of formula (Ig) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Ig) is a compound of formula (Ig) wherein:
R¹ and R² are both —R⁶—S—C(═NR⁴)N(R⁴)R⁵;
each R⁴ and R⁵ is independently hydrogen, alkyl, or —OR⁷;
R⁶ is a direct bond or a straight or branched alkylene chain;
R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

Another embodiment of the compounds of formula (Ig) is the compound of formula (Ig) that is (4-amino-4H-1,2,4-triazole-3,5-diyl)bis(methylene) dicarbamimidothioate.

Of the compounds of formula (I) described above in the Summary of the Invention, one embodiment is wherein the compound of formula (I) is a compound of formula (Ih):

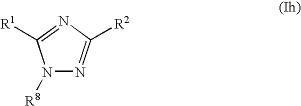

wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
R⁸ is independently hydrogen or alkyl.

One embodiment of the compounds of formula (Ih) is a compound of formula (Ih) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
R⁸ is independently hydrogen or alkyl.

Another embodiment of the compounds of formula (Ih) is a compound of formula (Ih) wherein:
R¹ and R² are both —R⁶—S—C(═NR⁴)N(R⁴)R⁵;
each R⁴ and R⁵ is independently hydrogen, alkyl, or —OR⁷;
R⁶ is a direct bond or a straight or branched alkylene chain;
R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
R⁸ is independently hydrogen or alkyl.

Another embodiment of the compounds of formula (Ih) is the compound of formula (Ih) that is (1H-1,2,4-triazole-3,5-diyl)bis(methylene)dicarbamimidothiodate.

Another aspect of the invention are methods for treating an iron disorder in a mammal, preferably a human, or a disease or condition associated with an iron disorder in a mammal, preferably a human, wherein the method comprises administering to the mammal in need thereof a therapeutically effective amount of a compound of the invention, as set forth above in the Summary of the Invention, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above in the Summary of the Invention, as a stereoisomer, enantiomer, tautomer thereof or mixtures thereof, or a pharmaceutically acceptable salt, solvate or prodrug thereof, and a pharmaceutically acceptable excipient.

One embodiment of this aspect is where the disease or condition associated with the iron disorder is due to an accumulation of iron in the body tissues of the mammal.

Another embodiment of this aspect is where the iron disorder is a primary iron overload disorder.

Of this embodiment, a preferred embodiment is where the primary iron overload disorder is independently selected from the group consisting of hereditary hemochromatosis, juvenile hemochromatosis, ferroportin disease, neonatal hemochromatosis, Bantu siderosis, African iron overload, gracile syndrome, ataxia, and Friedreich Ataxia. A more preferred embodiment is where the primary iron overload is hereditary hemochromatosis.

Another embodiment of this aspect is where the iron disorder is a secondary iron overload disorder.

Another embodiment of this aspect is where the iron disorder is transfusional iron overload disorder.

Another embodiment of this aspect is where the disease or condition is independently selected from the group consisting of thalassemia (beta and alpha, major, minor and intermedia), hypochromic microcytic anemia, sickle cell anemia, microcytic iron loading anemia, hereditary sideroblastic anemia, congenital dyserythropoeitic anemia, porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

Another embodiment of this aspect is where the disease or condition associated with an iron overload is independently selected from the group consisting of neurodegenerative disease (including ALS, prion diseases, Parkinson's, and Alzheimers), cardiovascular disease (including atherosclerosis, ischemic cerebrovascular disease and ischemic stroke), inflammation (including arthritis and disease progression in viral hepatitis), cancer, insulin resistance, non-alcoholic liver disease, alcoholic liver disease, and infectious disease (including HIV, malaria and Yersinia infections).

Another embodiment of the invention are methods for treating an iron disorder associated with DMT1 activity in a mammal, preferably a human, or for treating a disease or condition associated with DMT1 activity in a mammal, preferably a human, wherein the method comprises administering to the mammal in need thereof a therapeutically effective amount of a compound of the invention, as set forth above in the Summary of the Invention, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above in the Summary of the Invention, as a stereoisomer, enantiomer, tautomer thereof or mixtures thereof, or a pharmaceutically acceptable salt, solvate or prodrug thereof, and a pharmaceutically acceptable excipient.

Of this embodiment, one embodiment is where the DMT1 activity is upregulated (i.e., increased levels of DMT1 activity as compared to normal levels of DMT1 activity).

Of this embodiment, another embodiment is where the therapeutically effective amount administered to the mammal is a DMT1-inhibitory amount.

Specific embodiments of the compounds of the invention are described in more detail below in the following sections.

### Utility and Testing of the Compounds of the Invention

The present invention is directed to compounds and pharmaceutical compositions comprising the compounds, as described herein and above in the Summary of the Invention, which are useful in the treatment of iron disorders in a mammal, preferably a human, by modulating, preferably inhibiting, DMT1 activity.

The term “iron disorder” refers to a condition in a mammal, preferably a human, wherein the level of iron in the body is outside the normal range for the particular mammal (i.e. abnormal iron level), such as an elevated or a decreased iron serum level compared to the normal iron serum level for the mammal or an increased or decreased level of iron in the liver of the mammal as compared to the normal level of iron in the liver in the mammal. Abnormal iron serum levels can be determined by direct measurement of serum iron using a colorimetric assay, or by the standard transferrin saturation assay (which reveals how much iron is bound to the protein that carries iron in the blood), or by the standard serum ferritin assay. For example, transferrin saturation levels of 45% or higher are usually indicative of abnormally high levels of iron in the serum. Abnormal iron levels in the liver can be determined measuring the iron content of the liver from tissue obtained by a liver biopsy or by imaging technique such as MRI and/or SQUID. The degree of iron levels in other tissues (e.g., brain, heart) may also be estimated using these and other imaging techniques. Preferably, for purposes of this invention, an abnormal iron level is an elevated iron level in serum or tissue.

The term “iron disorders” therefore includes both iron deficiency disorders and iron overload disorders. Preferably, the iron disorder is an iron overload disorder, such as primary iron overload disorder (including, but not limited to, hereditary hemochromatosis, juvenile hemochromatosis, ferroportin disease, neonatal hemochromatosis, Bantu siderosis, African iron overload, gracile syndrome, ataxia, and Friedreich Ataxia, as well as all of the anemias listed below in which patients may not be transfused but may become iron overloaded due to increased erythroid drive and the resulting increased iron absorption in the gut) and secondary (or transfusional) iron overload disorder which can be caused by repeated transfusions used to treat a number of distinct anemias, including, but not limited to, thalassemia (beta and alpha, major, minor and intermedia), hypochromic microcytic anemias, sickle cell anemia, microcytic iron loading anemias, hereditary sideroblastic anemias, congenital dyserythropoeitic anemias, porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

Iron disorders of particular interest in the practice of the invention are iron overload disorders where the level of iron in a mammal is higher than the normal level of iron in the mammal. Such iron overload disorders including, but are not limited to, primary iron overload disorders (including, but not limited to, hereditary hemochromatosis, juvenile hemochromatosis, ferroportin disease, neonatal hemochromatosis, Bantu siderosis, African iron overload, gracile syndrome, ataxia, and Friedreich Ataxia, as well as all of the anemias listed below, in which patients may not be transfused but may become iron overloaded due to increased erythroid drive and the resulting increased iron absorption in the gut), and secondary (transfusional) iron overload disorders (including, but not limited to, thalassemia (beta and alpha, major, minor and intermedia)), hypochromic microcytic anemias, sickle cell anemia, microcytic iron loading anemias, hereditary sideroblastic anemias, congenital dyserythropoeitic anemias, porphyria cutanea tarda, pyruvate kinase deficiency, hereditary atransferrinemia, ceruloplasmin deficiency, myelodysplastic syndromes, pulmonary hemosiderosis, aceruloplasminemia, and x-linked sideroblastic anemia. Iron overload may also be responsible for a portion of the pathology observed in neurodegenerative diseases (including ALS, prion diseases, Parkinson's, Alzheimers), cardiovascular diseases (including atherosclerosis, ischemic cerebrovascular disease and ischemic stroke), inflammatory diseases and conditions (including arthritis and disease progression in viral hepatitis), cancer, insulin resistance, non-alcoholic liver disease, alcoholic liver disease, and infectious disease (including HIV, malaria and Yersinia infections).

The compounds of the invention, and pharmaceutical compositions comprising the compounds of the invention, are useful in treating iron disorders by modulating, preferably inhibiting, DMT1 activity. There is evidence that the upregulation (i.e., increased activity) of DMT1 has a role in iron disorders caused by genetic abnormalities, such as hereditary hemochromatosis. Hereditary hemochromatosis is an iron overload disorder due to intestinal iron hyperabsorption. Hereditary hemochromatosis is characterized by a slow accumulation of iron from the diet to toxic levels resulting in tissue injury and multi-organ malfunction. Patients, typically men, develop symptoms of hemochromatosis in their fourth and fifth decade with variable combinations of cirrhosis, hepatoma, arthritis, hypogonadism, diabetes mellitus and cardiomyopathy. The biochemical profile shows elevated transferrin saturation above 45% and a high serum ferritin. The underlying genetic defect in hereditary hemochromatosis is a mutation in the hemochromatosis gene (HFE) on chromosome 6p21. 90% of Northern Europeans with hereditary hemochromatosis are homozygous for a single missense mutation, C282Y in exon 4 of the HFE gene.

DMT1 activity has also been implicated in the etiology and pathophysiology of hypochromic microcytic anemias, thalassemia, microcytic iron loading anemias, hereditary sideroblastic anemias, hereditary hypochromic anemias, congenital dyserythropoietic anemias, pyruvate kinase deficiency, hereditary atransferrinemia, and certain myelodysplastic syndromes, as there is a direct correlation between the degree of iron limited anemia, increased DMT1 expression in the duodenum and, by extension, increased iron absorption via DMT1 (Morgan et al., Blood Cells Molecules and Diseases, 2002, 29:384-399).

There is also evidence that DMT1 has a role in iron disorders such as acquired iron overload. The risk factors for acquired iron overload might include for example excessive ingestion of red meat, iron supplements or foods that are iron fortified. Acquired iron overload can also occur from the use of iron cookware, drinking unpurified tap water, use of oral contraceptives, blood transfusions and cigarette smoking. DMT1 pattern of expression and function supports it as a candidate target for the treatment of acquired iron overload and other related maladies.

In addition to the small intestine, DMT1 is also highly expressed in the kidney suggesting a role in renal iron handling and possibly reabsorption of filtered iron (Ferguson et al., Am. J. Physiol. Renal. Physiol., 2001, 280: F803-F814) and is also involved in the delivery of iron to peripheral tissues by transferrin (Fleming et al., Proc. Natl. Acad. Sci., 1998, 85:1148-1153). DMT1 inhibitors, when dosed in a fashion that increases their systemic exposure, may be useful in an acute unloading of iron via the urine, by inhibiting DMT1 expressed in the kidney.

DMT1 may also play a role in regulating iron flux to the brain. As there is some indication that iron overload in the brain may play a role in brain pathology, such as Alzheimer's, DMT1 inhibitors may act to reduce the amount of iron absorbed by the brain, when dosed in a fashion that increases their systemic exposure and allows them to play a role at the blood brain barrier or within the brain (Lehmann et al, 2006, J. Med. Genet., 2006, 43(10):e52; Schenck et al., Top. Magn Reson. Imaging., 2006, 17(1):41-50).

Studies show that mutant mice that are defective in DMT1 activity (mk/mk) develop hyprochromic microcytic anemia, a severe form of iron deficiency anemia, due to a defect in intestinal iron absorption. In contrast, the hfe^{-/-} knockout mouse model of hereditary hemochromatosis is characterized by an enhanced intestinal iron uptake and total body iron overload. The hfe^{-/-}:mk/mk double mutant mouse, which carries mutations in both the HFE and DMT1 genes, fails to load iron, indicating that hemochromatosis (hfe^{-/-}) can be prevented by blocking the flux of iron through the DMT1 protein (Levy et al., J. Clin. Invest., 2000, 105:1209-16). In addition, studies of human patients with hereditary hemochromatosis show that DMT1 is inappropriately upregulated at the intestinal brush border. This aberrant excessive expression of DMT1 in hereditary hemochromatosis is fundamental to the primary pathophysiology of this condition (Zoller et al., Gastroenterology, 2001, 120:1412-1419). These findings have made DMT1 a therapeutic target for the treatment of iron overload disorders in general, and, in particular, for the treatment of hereditary hemochromatosis. In further support of DMT1 as a therapeutic target in the treatment of iron overload, it has been shown in clinical studies that the majority of the excess iron burden is absorbed in the form of ferrous (non-heme) iron, as opposed to heme-iron (Lynch et al., Blood, 1989, 74:2187-2193).

While not wishing to be bound to any particular mechanism of action, the compounds of the invention, and pharmaceutical compositions comprising the compounds of the invention, are useful in treating iron disorders by directly interacting with a region of the DMT1 protein that modulates or controls iron flux. A direct interaction is supported by the fact that the compounds are not potent inhibitors of cation flux in the closely related transporter Natural Resistance-Associated Macrophage Protein-1 (NRAMP1). In general, the compounds of the invention modulate the activity of DMT1 downwards, thereby inhibiting the ability of DMT1 to uptake non-heme iron across the cellular membrane. The compounds of the invention are therefore considered to be DMT1 inhibitors and are therefore useful in treating iron disorders which are ameliorated by the modulation, preferably the inhibition, of DMT1 activity. The compounds of the invention, as DMT1 inhibitors, are also useful in reducing normal or slightly abnormal iron serum levels in a mammal, preferably a human, wherein the reduction of iron serum levels provides a therapeutic benefit to the mammal, preferably a human, such as neuroprotective activity after a stroke.

The compounds of the invention, and pharmaceutical compositions comprising the compounds of the invention, are also useful in treating or preventing symptoms, diseases and/or conditions in a mammal associated with hereditary hemochromatosis due to accumulation of iron in body tissues such as arthritis, liver disease, heart disease, impotence, early menopause, abnormal skin pigmentation, thyroid deficiency, damage to pancreas, diabetes, and damage to adrenal gland (Sheth et al, Annu. Rev. Med., 2000, 51:443-464).

The compounds of the invention, and pharmaceutical compositions comprising the compounds of the invention, are also useful in treating or preventing other forms of hemochromatosis including, but are not limited to, juvenile hemochromatosis and neonatal hemochromatosis. Juvenile hemochromatosis has a much earlier onset and exhibits more severe symptoms such as endocrine dysfunction, joint disease, and cardiac abnormalities due to excessive iron deposition from an early age. Neonatal hemochromatosis is a rare fetal gestational condition that results in iron accumulation in the liver of the fetus.

The compounds of the invention, and pharmaceutical compositions comprising the compounds of the invention, are also useful in treating or preventing transfusional iron overload. Chronic blood transfusion is the established therapy for thalassaemia major, bone marrow failure and complications of sickle cell anaemia and other related disorders. With hypertransfusion, the systemic iron load accumulates. Because there is no natural way for the body to eliminate the iron, the excess iron in the transfused blood builds up to cause iron overload and becomes toxic to tissues and organs, particularly the liver, heart, and pancreas. Transfusional iron overload typically results in the patient's premature death from organ failure. The transfusional iron overload is unfortunately augmented by increased iron absorption, which is the natural attempt of the body to increase iron levels in order to promote erythropoiesis, which is itself compromised by the disease states above. Decreased absorption of iron by the inhibition of DMT1 activity may reduce the iron overload related to the transfusional iron overload and supports the use of DMT1 inhibitors for the treatment of this disease.

In addition, due to iron's ability to generate reactive oxygen species (free radicals), which can result in inflammation and tissue damage, the compounds of the invention, and pharmaceutical compositions comprising the compounds of the invention, may also be useful as anti-inflammatory or neuroprotective agents due to their ability to reduce iron serum levels by the modulation, preferably inhibition, of DMT1 activity.

The general value of the compounds of the invention, and pharmaceutical compositions comprising the compounds of the invention, in modulating, preferably inhibiting, DMT1 activity can be determined using the assays described herein or below in the Biological Assays section. Alternatively, the general value of the compounds of the invention, and pharmaceutical compositions comprising the compounds of the invention, in treating iron disorders in humans may be established in industry standard animal models for demonstrating the efficacy of compounds in treating iron disorders.

In particular, identification of the compounds of the invention ability to modulate, preferably to inhibit, DMT1 activity, can be assessed using a variety of in vitro and in vivo assays, for measuring uptake of reduced iron (Fe²⁺). One such protocol involves the screening of chemical agents for ability to modulate the activity of DMT1 thereby identifying it as a modulating agent. The in vitro activity of DMT1 can be measured in cell based assays by either directly measuring iron flux (using a radioactively labelled iron ⁵⁵Fe) or by measuring the fluorescence of a cell permeable iron fluorophore such as calcein. Stable cell lines overexpressing DMT1 are exposed to ⁵⁵Fe or loaded with calcein and then compound is applied. Decreased flux of ⁵⁵Fe or lack of fluorescence quenching indicates that the given modulator has inhibited DMT1 function (Picard et al., J. Biol. Chem., 2000, 275(46):35738-45 and Wetli et al., Chem. Biol. 2006 September; 13(9):965-72). Alternatively, in another format electrophysiological techniques can be used to measure the current or iron or other metals traversing the cell membrane with DMT1 in a Xenopus oocyte or other cell based system (Gunshin et al., Nature, 1997, 31; 388(6641):482-8).

Other assays may involve intestinal cells or tissues which express endogenous DMT1, using the same detection techniques such as fluorescence, radiolabelled iron or electrophysiology. A human Caco2 cell line can be used for such assays (Alvarez-Hernandez et al., Biochimica. et. Biophysica. Acta., 1991, 1070:205-208). These assays can be performed in the presence of desferroxamine to render the cells iron deficient and upregulate DMT1 expression. Alternatively, intestinal tissue may be used, either as gut rings which will take up iron (Raja et al., Cell. Biochemistry and Function, 1987, 5:69-76; Leppert et al., J. of Pharm. Sci., 1994, 83:976-981), or as gut slices ex vivo (Vaghefi et al., Reprod. Nutr. Dev., 1998, 38:559-566) where iron flux across the epithelial layer can be assessed in an Ussing chamber. In these assays, tissue can be excised from iron replete or iron deficient animals. In addition, the heme versus non-heme iron absorptive capacity of the tissue can be measured.

These assays can be carried out in transfected cells, or cell or tissue endogenously expressing the channel of interest in a natural endogenous setting or in a recombinant setting. Other methods of testing the compounds disclosed herein are also readily known and available to those skilled in the art.

Compounds of the invention can also be tested in a variety of in vivo models so as to determine if they alleviate a particular iron disorder in a mammal, particularly an iron overload disorder, with minimal adverse events. The assays described herein and below in the Biological Assays Section are useful in assessing the in vivo activity of the compounds of the invention.

For example, a typical rat model of iron overload disorder can be created by establishing an iron deficient state in the rate, which will then cause the upregulation of DMT1 expression and activity, resulting in increased iron absorption. These models can be used to demonstrate that compounds of the invention have the ability to modulate, preferably inhibit, the activity of DMT1 as demonstrated by the increase in serum iron levels in the iron-deficient rat. Iron deficiency is induced in these rat models in order to mimic the DMT1 over-expression and iron hyperabsorption observed in humans having iron overload disorders such as hereditary hemochromatosis as well as humans suffering from thalassemia.

Alternatively, an iron deficient, and therefore hyperabsorptive state, may be induced by dietary means, such as, for example, treatment with phenylhydrazine, or by phlebotomy (Refino et al., Am. J. Clin. Nutr. 1983, 37:904-909; Redondo et al., Lab. Animal Sci. 1995, 45:578-583; Frazer et al., Gastroenterology, 2002, 123:835-844). Alternatively, iron absorption can also be stimulated by creating an hypoxic state to stimulate erythropoiesis (Raja et al., Br. J. Haematol., 1988, 68:373-378). In these models, a compound's efficacy can be assessed by measuring reduced iron flux via the duodenum acutely or by monitoring whether chronic exposure to a compound causes a decrease in the amount of iron loading as measured by serum iron, transferrin saturation, ferritin and liver iron. Alternatively, iron flux in these animals can be measured by tracing the absorption of radioactive iron administered orally. These experiments can also be performed in iron replete animals, although changes in these parameters will be less pronounced and therefore compound efficacy will be more difficult to judge.

Genetic rat models of iron overload offers another format to show efficacy of DMT1 inhibitors in preventing further iron loading. These models are applicable to a variety of iron disorders such as hereditary hemochromatosis (Levy et al., Blood, 1999, 94:9-11), juvenile hemochromatosis (Huang et al., J. Clin. Invest., 2005 115:2187-2191), beta-2-microglobulin (de Sousa et al., Immun. Lett., 1994, 39:105-111), thalassemia (Ciavatta et al., Proc. Nat. Acad. Sci., 1995, 92: 9259-9263), hypotransferrinmia (Craven et al., Proc. Nat. Acad. Sci., 1987, 84(10):3457-61) and other hypochromic microcytic anemias. A compound's efficacy can be assessed by measuring reduced iron flux via the duodenum acutely or by monitoring whether chronic exposure to a compound causes a decrease in the amount of iron loading as judged by serum iron, transferrin saturation, ferritin and liver iron. Alternatively, iron flux in these animals can be measured by tracing the absorption of radioactive iron administered orally.

Typically, a successful therapeutic agent of the present invention will meet some or all of the following criteria. Oral availability should be at less than 5%. Animal model efficacy is less than about 0.1 μg to about 100 mg/Kg body weight and the target human dose is between 0.1 μg to about 100 mg/Kg body weight, although doses outside of this range may be acceptable (“mg/Kg” means milligrams of compound per kilogram of body mass of the subject to whom it is being administered). The therapeutic index (or ratio of toxic dose to therapeutic dose) should be greater than 100. The potency (as expressed by IC₅₀ value) should be less than 10 μM, preferably below 1 μM and most preferably below 50 nM. The IC₅₀ (“Inhibitory Concentration 50%”) is a measure of the amount of compound required to achieve 50% inhibition of DMT1, over a specific time period, in an assay of the invention.

In another use of the invention, the compounds of the invention can be used in in vitro or in vivo studies as exemplary agents for comparative purposes to find other compounds useful in the treatment of an iron disorder or diseases or conditions associated with an iron disorder.

In another use of the invention, the compounds of the invention can be used in the preparation of a medicament for the treatment of an iron disorder in a mammal or for the treatment of a disease or condition associated with an iron disorder in a mammal.

### Pharmaceutical Compositions of the Invention and Administration

The present invention also relates to pharmaceutical composition containing the compounds of the invention disclosed herein. In one embodiment, the present invention relates to a composition comprising compounds of the invention in a pharmaceutically acceptable carrier, excipient or diluent and in an amount effective to modulate, preferably inhibit, DMT1 in order to treat iron disorders when administered to an animal, preferably a mammal, most preferably a human patient.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 0.001 mg/Kg (i.e., 0.07 mg) to about 100 mg/Kg (i.e., 7.0 g); preferably a therapeutically effective dose is (for a 70 Kg mammal) from about 0.01 mg/Kg (i.e., 0.7 mg) to about 50 mg/Kg (i.e., 3.5 g); more preferably a therapeutically effective dose is (for a 70 Kg mammal) from about 1 mg/Kg (i.e., 70 mg) to about 25 mg/Kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodman et al., eds., Goodman and Gilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the invention can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the invention to target area, e.g., skin surfaces, mucous membranes, and the like, which are adjacent to peripheral neurons which are to be treated. This amount will generally range from about 0.0001 mg to about 1 g of a compound of the invention per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present invention as desired.

The compositions of the invention can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al, Regional Anesthesia 22 (6): 543-551 (1997), all of which are incorporated herein by reference.

The compositions of the invention can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as described in PCT Patent No. WO 98/50016.

Current methods for ocular delivery include topical administration (eye drops), subconjunctival injections, periocular injections, intravitreal injections, surgical implants and iontophoresis (uses a small electrical current to transportionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (oral, intravenous, inhalation, sub-cutaneous, rectal etc.), dosage forms, suitable pharmaceutical excipients and other matters relevant to the delivery of the compounds to a subject in need thereof.

### Combination Therapy

The compounds of the invention may be usefully combined with one or more other compounds of the invention or one or more other therapeutic agent or as any combination thereof, in the treatment of iron disorders. For example, a compound of the invention may be administered simultaneously, sequentially or separately in combination with other therapeutic agents, including, but not limited to iron chelators, e.g. deferasirox (ICL-670), deferiprone, and desferroxamine, and erythropoietin (EPO), e.g. rh-EPO. In addition, compounds of the invention, as inhibitors of DMT1 activity, could also be combined with phlebotomy therapy for the treatment of iron overload disorders.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Kits-of-Parts

The present invention also provides kits that contain a pharmaceutical composition which includes one or more compounds of the invention. The kit also includes instructions for the use of the pharmaceutical composition for treating iron disorders as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### Preparation of the Compounds of the Invention

The following Reaction Schemes illustrate methods to make compounds of the invention, i.e., compounds of formula (I):

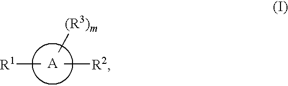

wherein m,

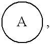

R¹, R² and R³ are as defined above in the Summary of the Invention for compounds of formula (I), as a stereoisomer, enantiomer, tautomer thereof or mixtures thereof; or a pharmaceutically acceptable salt, solvate or prodrug thereof.

In particular, the following Reaction Schemes illustrate methods to make compounds of formula (Ia), compounds of formula (Ib), compounds of formula (Ic), compounds of formula (Id), compounds of formula (Ie), compounds of formula (If), compounds of formula (Ig) and compounds of formula (Ih) as described above in the Embodiments of the Invention. These compounds are compounds of formula (I), as set forth above in the Summary of the Invention. It is understood that one skilled in the art would be able to make these compounds by similar methods or by methods known to one skilled in the art. It is also understood that one skilled in the art would be able to make in a similar manner as described below other compounds of the invention not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Smith, M. B. and J. March, Advanced Organic Chemistry: Reactions, Mechanisms, and Structure, 5th edition (Wiley, December 2000)) or prepared as described herein.

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters.

Protecting groups may be added or removed in accordance with standard techniques, which are known to one skilled in the art and as described herein.

The use of protecting groups is described in detail in Greene, T. W. and P. G. M. Wuts, Greene's Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

The starting materials for the reaction schemes described below are commercially available or can be prepared according to methods known to one skilled in the art or by methods disclosed herein.

### A. Preparation of Compounds of Formula (Ia-1)

Compounds of formula (Ia-1) are compounds of formula (Ia), as set forth above in the Embodiments of the Invention, where R¹ and R² are each —R⁶—S—C(═NR⁴)N(R⁴)R⁵, each R⁴ and each R⁵ are hydrogen and each R⁶ is —CH₂— and Q, R^{ab}, R^{3c} and R^{ad} are each as described above in the Embodiments of the Invention for compounds of formula (Ia), and X is halo, preferably bromo or chloro, and are prepared as set forth below in Reaction Scheme 1.

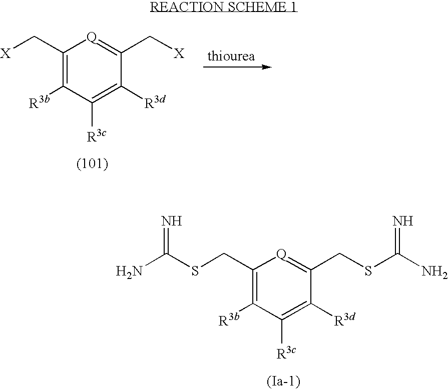

The starting materials for the above reaction scheme are commercially available or can be prepared according to methods known to one skilled in the art or by methods disclosed herein. In general, the compounds of formula (Ia-1) are prepared in the above reaction scheme as follows:

The displacement of halogen groups of the compound of formula (101) with thiourea under standard conditions known to one skilled in the art affords the compound of formula (Ia-1) of the invention.

Alternatively, the compounds of formula (Ia-1), as set forth above, can be prepared as set forth below in Reaction Scheme 2.

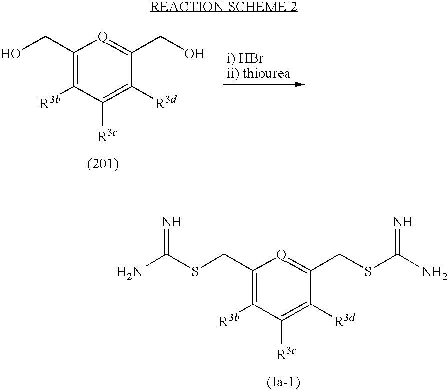

The starting materials for the above reaction scheme are commercially available or can be prepared according to methods known to one skilled in the art or by methods disclosed herein. In general, the compounds of the invention are prepared in the above reaction scheme as follows:

A compound of formula (201) is treated with HBr and subsequently with thiourea under standard conditions known to one skilled in the art to afford the compound of formula (Ia-1) of the invention.

### B. Preparation of Compounds of Formula (Ia-2)

Compounds of formula (Ia-2) are compounds of formula (Ia), as set forth above in the Embodiments of the Invention, where R′ and R² are each —R⁶—N(R⁷)C(═NR⁴)N(R⁴)R⁵, each R⁴ and each R⁷ are hydrogen, each R⁵ is —CN and each R⁶ is —CH₂—, and Q, R^{3b}, R^{3c} and R^{3d} are each as described above in the Embodiments of the Invention for compounds of formula (Ia), and X is halo, preferably bromo or chloro, and are prepared as set forth below in Reaction Scheme 3.

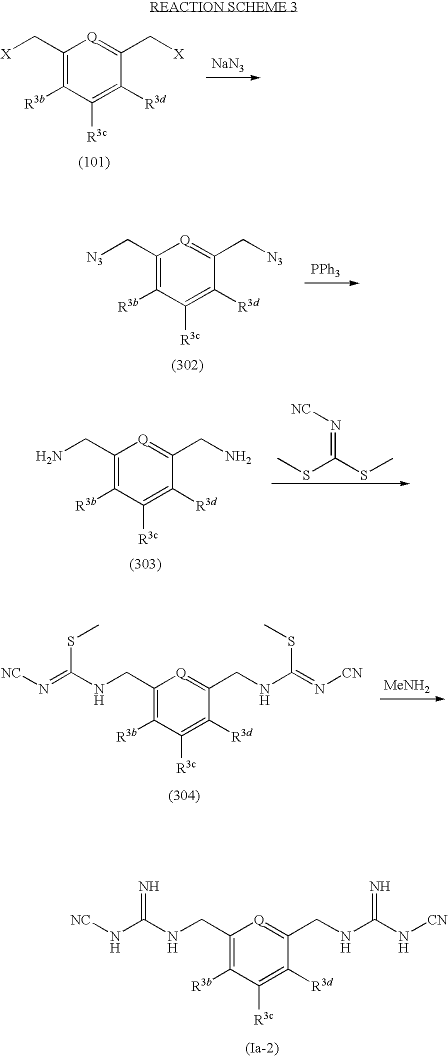

The starting materials for the above reaction scheme are commercially available or can be prepared according to methods known to one skilled in the art or by methods disclosed herein. In general, the compounds of formula (Ia-2) are prepared in the above reaction scheme as follows:

Displacement of the halogen groups of a compound of formula (101) with sodium azide affords an azide compound of formula (302), which, upon reduction with a suitable reducing agent such as, but not limited to, triphenylphosphine, yields a diamino compound of formula (303). Sequential treatment of the diamino compound of formula (303) with dimethyl N-cyanodithioiminocarbonate followed by methylamine affords a compound of formula (Ia-2) of this invention.

### C. Preparation of Compounds of Formula (Ia-3)

Compounds of formula (Ia-3) are compounds of formula (Ia), as set forth above in the Embodiments of the Invention, where R¹ and R² are —R⁶—C(═NR⁴)N(R⁴)R⁵, each R⁴ and each R⁵ are hydrogen and each R⁶ is —CH₂— and Q, R^{3b}, R^{3c} and R^{3d} are each as described above in the Embodiments of the Invention for compounds of formula (Ia), and are prepared as set forth below in Reaction Scheme 4.

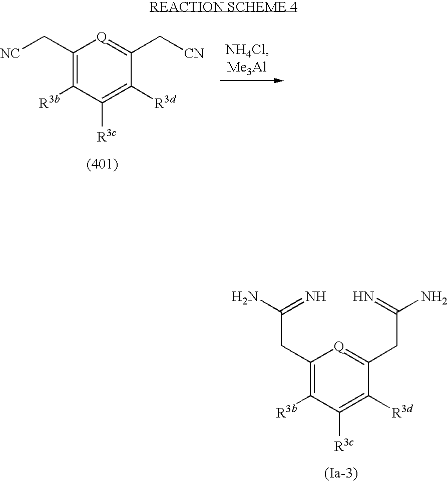

The starting materials for the above reaction scheme are commercially available or can be prepared according to methods known to one skilled in the art or by methods disclosed herein. In general, the compounds of formula (Ia-3) are prepared in the above reaction scheme as follows:

A cyano compound of formula (401) is treated with ammonium chloride and trimethylaluminum under conditions known to one skilled in the art to afford a compound of formula (Ia-3) of this invention.

### D. Preparation of Compounds of Formula (Ia-4)

Compounds of formula (Ia-4) are compounds of formula (Ia), as set forth above in the Embodiments of the Invention, where R¹ and R² are —R⁶—N(R⁷)C(═NR⁴)N(R⁴)R⁵, R⁴, R⁵ and R⁷ are hydrogen and R⁶ is —CH₂— and Q, R^{3b}, R^{3c} and R^{3d} are each as described above in the Embodiments of the Invention for compounds of formula (Ia), and X is halo, preferably bromo or chloro, and are prepared as set forth below in Reaction Scheme 5.

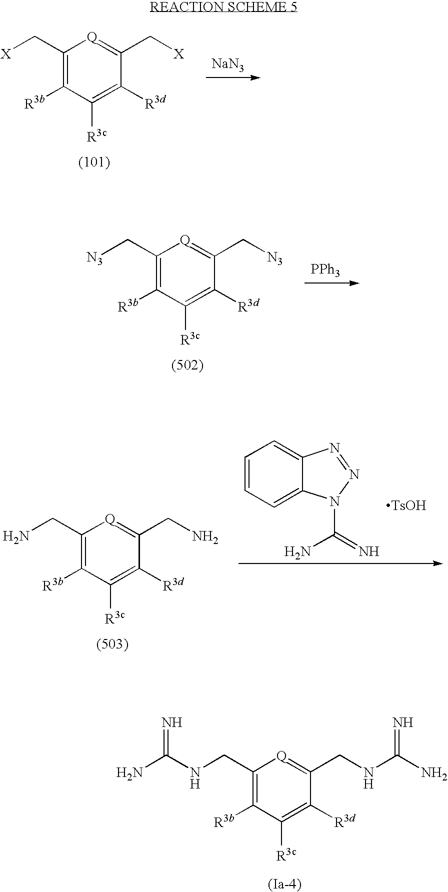

The starting materials for the above reaction scheme are commercially available or can be prepared according to methods known to one skilled in the art or by methods disclosed herein. In general, the compounds of formula (Ia-4) are prepared in the above reaction scheme as follows:

Displacement of the halogen groups of a compound of formula (101) with sodium azide affords an azide compound of formula (502), which upon reduction with a suitable reducing agent such as, but not limited to, triphenylphosphine yields a diamino compound of formula (503). Treatment of the diamino compound of formula (503) with 1-benzotriazole-carboxamidinium tosylate in a suitable solvent such as, but not limited to, N,N-dimethylformamide in the presence of a suitable base such as, but not limited to, N,N-diisopropylethylamine affords a compound of formula (Ia-4) of the invention.

### E. Preparation of Compounds of Formula (Ia-5)

Compounds of formula (Ia-5) are compounds of formula (Ia), as set forth above in the Embodiments of the Invention, where R¹ and R² are —R⁶—S—C(═NR⁴)N(R⁴)R⁵, R⁴ and R⁵ are hydrogen, R⁶ is a direct bond and Q, R^{3b}, R^{3c} and R^{3d} are each as described above in the Embodiments of the Invention for compounds of formula (Ia), and are prepared as set forth below in Reaction Scheme 6.

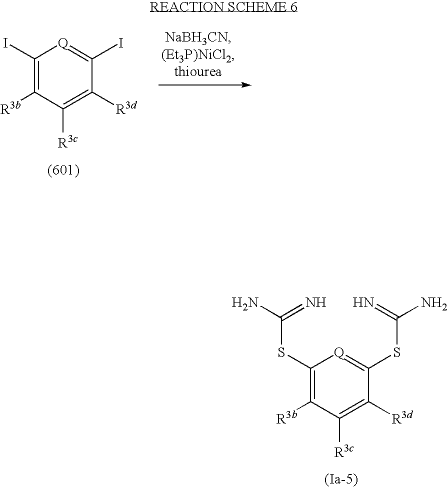

The starting materials for the above reaction scheme are commercially available or can be prepared according to methods known to one skilled in the art or by methods disclosed herein. In general, the compounds of formula (Ia-5) are prepared in the above reaction scheme as follows:

An aryl diiodide of formula (601) is treated with thiourea, a low-valent nickel complex formed from bis(triethylphosphine)nickel(II) chloride and a suitable reductant, such as, but not limited to, sodium cyanoborohydride, to afford a compound of formula (Ia-5) of the invention.

### F. Preparation of Compounds of Formula (Ib-1)

Compounds of formula (Ib-1) are compounds of formula (Ib), as set forth above in the Embodiments of the Invention, where R′ and R² are each —R⁶—S—C(═NR⁴)N(R⁴)R⁵, each R⁴ and each R⁵ are hydrogen and each R⁶ is —CH₂— and R^{3a}, R^{3b}, R^{3c} and R^{3d} are each as described above in the Embodiments of the Invention for compounds of formula (Ib), and X is halo, preferably bromo or chloro, and are prepared as set forth below in Reaction Scheme 7.

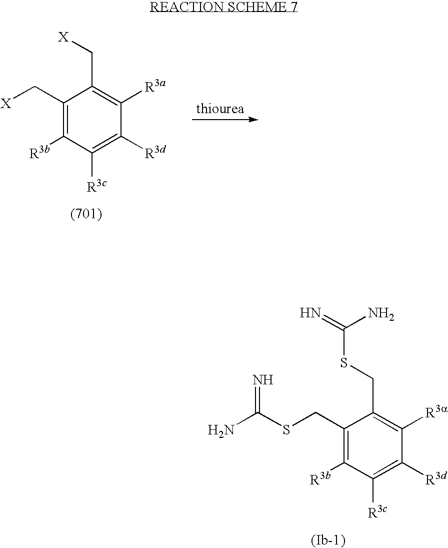

The starting materials for the above reaction scheme are commercially available or can be prepared according to methods known to one skilled in the art or by methods disclosed herein. In general, the compounds of formula (Ib-1) are prepared in the above reaction scheme as follows:

The displacement of halogen groups of the compound of formula (701) with thiourea under conditions known to one skilled in the art affords the compound of formula (Ib-1) of the invention.

### G. Preparation of Compounds of Formula (Ic-1)

Compounds of formula (Ic-1) are compounds of formula (Ic), as set forth above in the Embodiments of the Invention, where R¹ and R² are —R⁶—S—C(═NR⁴)N(R⁴)R⁵, each R⁴ and each R⁵ are hydrogen, each R⁶ is —CH₂— and R₃ₐ, R^{3b}, R^{3c} and R^{3d} are each as described above in the Embodiments of the Invention for compounds of formula (Ic), and are prepared as set forth below in Reaction Scheme 8.

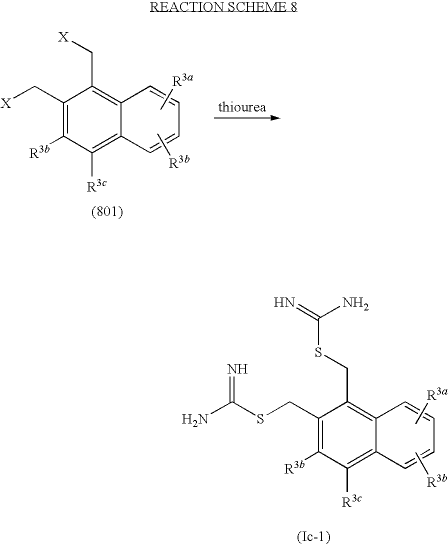

The starting materials for the above reaction scheme are commercially available or can be prepared according to methods known to one skilled in the art or by methods disclosed herein. In general, the compounds of formula (Ic-1) are prepared in the above reaction scheme as follows:

The displacement of halogen groups of the compound of formula (801) with thiourea under conditions known to one skilled in the art affords the compound of formula (Ic-1) of the invention.

### H. Preparation of Compounds of Formula (Id-1)

Compounds of formula (Id-1) are compounds of formula (Id), as set forth above in the Embodiments of the Invention, where R¹ and R² are each —R⁶—S—C(═NR⁴)N(R⁴)R⁵, each R⁴ and each R⁵ are hydrogen, each R⁶ is —CH₂ and R^{1a}, R^{3b}, R^{3c} and R^{3d} are each as described above in the Embodiments of the Invention for compounds of formula (Id), and X is halo, preferably bromo or chloro, and are prepared as set forth below in Reaction Scheme 9.

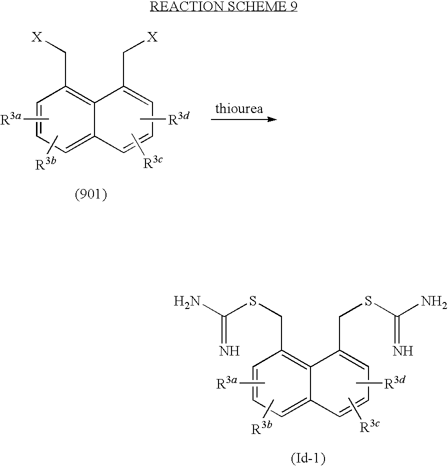

The starting materials for the above reaction scheme are commercially available or can be prepared according to methods known to one skilled in the art or by methods disclosed herein. In general, the compounds of formula (Id-1) are prepared in the above reaction scheme as follows:

The displacement of halogen groups of the compound of formula (901) with thiourea affords the compound of formula (Id-1) of the invention.

### I. Preparation of Compounds of Formula (Ie-1)

Compounds of formula (Ie-1) are compounds of formula (Ie), as set forth above in the Embodiments of the Invention, where R¹ and R² are —R⁶—S—C(═NR⁴)N(R⁴)R⁵, each R⁴ and each R⁵ are hydrogen and each R⁶ is —CH₂— and G, R^{3a} and R^{3b} are each as described above in the Embodiments of the Invention for compounds of formula (Ie), and X is halo, preferably bromo or chloro, and are prepared as set forth below in Reaction Scheme 10.

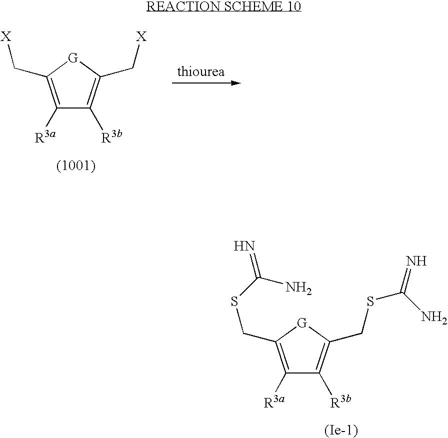

The starting materials for the above reaction scheme are commercially available or can be prepared according to methods known to one skilled in the art or by methods disclosed herein. In general, the compounds of formula (Ie-1) are prepared in the above reaction scheme as follows:

The displacement of halogen groups of the compound of formula (1001) with thiourea under conditions known to one skilled in the art affords the compound of formula (Ie-1) of the invention.

### J. Preparation of Compounds of Formula (If-1)

Compounds of formula (If-1) are compounds of formula (If), as set forth above in the Embodiments of the Invention, where R¹ and R² are —R⁶—S—C(═NR⁴)N(R⁴)R⁵, each R⁴ and each R⁵ are hydrogen and each R⁶ is —CH₂— and G¹, G², R^{3a} and R^{3b} are each as described above in the Embodiments of the Invention, and X is halo, preferably bromo or chloro, and are prepared as set forth below in Reaction Scheme 11.

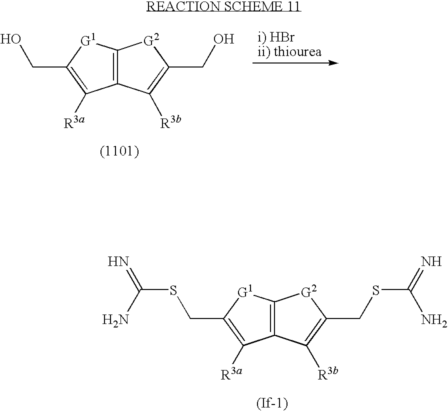

The starting materials for the above reaction scheme are commercially available or can be prepared according to methods known to one skilled in the art or by methods disclosed herein. In general, the compounds of formula (If-1) are prepared in the above reaction scheme as follows:

A compound of formula (1101) is treated with HBr and subsequently with thiourea under conditions known to one skilled in the art to afford the compound of formula (If-1) of the invention.

All compounds of the invention as prepared above and below which exist in free base or acid form may be converted to their pharmaceutically acceptable salt by treatment with the appropriate inorganic or organic base or acid by methods known to one skilled in the art. Salts of the compounds prepared herein may be converted to their free base or acid by standard techniques known to one skilled in the art.

The following Preparations, which are directed to the preparation of intermediates used in the preparation of the compounds of the invention, and the following Examples, which are directed to the preparation of the compounds of the invention, are provided as a guide to assist in the practice of the invention, and are not intended as a limitation on the scope of the invention.

### Preparation 1

### Preparation of (2,4,6-trimethyl-1,3-phenylene)dimethanamine

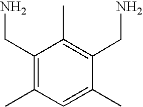

### A. Synthesis of 2,2′-(2,4,6-trimethyl-1,3-phenylene)bis(methylene)diisoindoline-1,3-dione

A mixture of 2,4-bis(chloromethyl)-1,3,5-trimethylbenzene (2.39 g, 11.00 mmol), potassium phthalimide (8.15 g, 44.00 mmol), potassium iodide (3.65 g, 22.00 mmol) and N,N-dimethylformamide (80 mL) was heated at 100° C. for 16 h. The reaction mixture was poured into water (300 mL) and the precipitate was collected by filtration and washed with water (50 mL). The resultant solid was triturated with boiling methanol (25 mL), air-dried and dried under high vacuum to afford 2,2-(2,4,6-trimethyl-1,3-phenylene)bis(methylene)diisoindoline-1,3-dione as a colorless solid in 63% yield (3.02 g): ¹H NMR (300 MHz, CDCl₃) δ 7.79-7.75 (m, 4H), 7.70-7.64 (m, 4H), 6.92 (s, 1H), 4.88 (s, 4H), 2.43 (s, 3H), 2.41 (s, 6H); MS (ES+) m/z 439.5 (M+1).

### B. Synthesis of (2,4,6-trimethyl-1,3-phenylene)dimethanamine

To a suspension of 2,2′-(2,4,6-trimethyl-1,3-phenylene)bis(methylene)diisoindoline-1,3-dione (3.02 g, 6.89 mmol) in anhydrous ethanol (20 mL) was added hydrazine monohydrate (3.6 mL, 74.0 mmol). The reaction mixture was heated at reflux for 5 h, cooled to ambient temperature and filtered. The filtrate was concentrated in vacuo to dryness to afford (2,4,6-trimethyl-1,3-phenylene)dimethanamine as a pale yellow solid in 96% yield (1.18 g): ¹H NMR (300 MHz, DMSO-d₆) δ 6.76 (s, 1H), 3.67 (s, 4H), 2.88 (br s, 4H), 2.35 (s, 3H), 2.26 (s, 6H); (ES+) m/z 179.4 (M+1).

### Preparation 2

### Preparation of dimethyl N,N′-(2,4,6-trimethyl-1,3-phenylene)bis(methylene)bis(N′-cyanocarbamimidothioate)

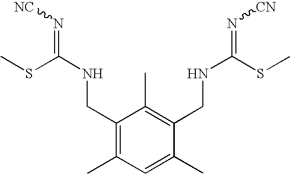

### A. Synthesis of 2,4-bis(azidomethyl)-1,3,5-trimethylbenzene

To a solution of 2,4-bis(chloromethyl)-1,3,5-trimethylbenzene (2.00 g, 9.21 mmol) in acetone (40 mL) was added sodium azide (1.32 g, 20.20 mmol) and the reaction mixture was heated at reflux for 6 h. Most of the acetone was removed on a rotary evaporator without heating. The resultant oily residue was diluted with diethyl ether (20 mL) and transferred to a separatory funnel. The organic phase was washed with water (3×20 mL) and brine (20 mL), dried over sodium sulfate, filtered and concentrated in vacuo to afford 2,4-bis(azidomethyl)-1,3,5-trimethylbenzene as a colorless oil which was used in the next step without purification: MS (ES+) m/z 231.3 (M+1).

### B. Synthesis of (2,4,6-trimethyl-1,3-phenylene)dimethanamine

To a solution of the crude 2,4-bis(azidomethyl)-1,3,5-trimethylbenzene in tetrahydrofuran (40 mL) and water (4 mL) was added triphenylphosphine (7.24 g, 27.60 mmol). The reaction mixture was stirred vigorously for 16 h at ambient temperature. The tetrahydrofuran was removed in vacuo and the residue was partitioned between 0.1 M aqueous hydrochloric acid (100 mL) and diethyl ether (50 mL) and transferred to a separatory funnel. The aqueous phase was washed with diethyl ether (2×50 mL) and carefully basified to pH ˜10 by the addition of a 10% aqueous solution of sodium carbonate. The aqueous phase was then extracted with dichloromethane (3×25 mL). The combined organic extracts were washed with brine (25 mL), dried over sodium sulfate, filtered and concentrated in vacuo to dryness to afford (2,4,6-trimethyl-1,3-phenylene)dimethanamine as a pale yellow solid in 38% yield over two steps (0.62 g): ¹H NMR (300 MHz, DMSO-d₆) δ 6.76 (s, 1H), 3.67 (s, 4H), 2.88 (br s, 4H), 2.35 (s, 3H), 2.26 (s, 6H); MS (ES+) m/z 179.4 (M+1).

### C. Synthesis of dimethyl N′,N-(2,4,6-trimethyl-1,3-phenylene)bis(methylene)bis(N′-cyanocarbamimidothioate)

To a solution of (2,4,6-trimethyl-1,3-phenylene)dimethanamine (0.62 g, 3.41 mmol) in anhydrous ethanol (15 mL) was added dropwise a solution of dimethyl N-cyanodithioiminocarbonate (90% purity, 1.02 g, 6.80 mmol) in anhydrous ethanol (15 mL). The resultant heterogeneous mixture was stirred for 16 h at ambient temperature. The precipitate was collected by filtration and air-dried. A 100 mg sample of this material was recrystallized from acetonitrile/water (1:1) to afford dimethyl N′,N-(2,4,6-trimethyl-1,3-phenylene)bis(methylene)bis(N′-cyanocarbamimidothioate) as a colorless solid (0.08 g): MS (ES+) m/z 375.6 (M+1)

### Preparation 3

### Preparation of 1,5-bis(bromomethyl)-2,4-diisopropylbenzene

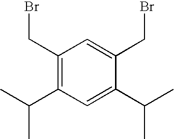

To a stirred solution of 1,3-diisopropylbenzene (2.50 mL, 13.20 mmol) and paraformaldehyde (1.40 g, 46.10 mmol) in acetic acid (8.0 mL) was added a solution of 33% hydrobromide in acetic acid (10 mL) at ambient temperature. The mixture was stirred at 130° C. for 15 h, poured into ice-water and filtered. The filtrate was neutralized with saturated sodium bicarbonate solution and extracted with dichloromethane (3×30 mL). The combined organic layers was dried over anhydrous sodium sulfate and filtered. The filtrate was concentrated in vacuo. The residue was purified by column chromatography eluted with hexane to afford 1,5-bis(bromomethyl)-2,4-diisopropylbenzene as a colorless solid in 43% yield (0.25 g). ¹H NMR (300 MHz, CDCl₃) δ 7.23 (s, 1H), 7.21 (s, 1H), 4.51 (s, 4H), 3.30-3.18 (m, 2H), 1.27 (d, J=6.8 Hz, 12H).

### Preparation 4

### Preparation of 1,1-(2,4,6-trimethyl-1,3-phenylene)diethanol

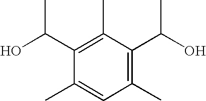

### A. Synthesis of 1,1′-(2,4,6-trimethyl-1,3-phenylene)diethanone

To a stirred suspension of aluminum trichloride (11.50 g, 86.24 mmol) in dichloromethane (15 mL) was added acetyl chloride (3.10 mL, 43.6 mmol) slowly under nitrogen atmosphere. The resulting reaction mixture was refluxed for 30 minutes, and mesitylene (2.00 mL, 14.40 mmol) in dichloromethane (8 mL) was added dropwise. The resulting reaction mixture was refluxed for 3 h, cooled to ambient temperature and poured into crushed ice. Dichloromethane (60 mL) was added and the two layers were separated. The aqueous layer was extracted with dichloromethane (60 mL). The combined organic layer was washed with saturated sodium bicarbonate solution (100 mL), brine (100 mL), dried over sodium sulfate, filtered and concentrated in vacuo to afford 1,1′-(2,4,6-trimethyl-1,3-phenylene)diethanone in a quantitative yield: ¹H NMR (300 MHz, CDCl₃) δ 6.88 (s, 1H), 2.45 (s, 6H), 2.21 (s, 6H), 2.11 (s, 31-1).

### B. Synthesis of 1,1′-(2,4,6-trimethyl-1,3-phenylene)diethanol

To a stirred solution of 1,1′-(2,4,6-trimethyl-1,3-phenylene)diethanone (1.00 g, 4.90 mmol) in tetrahydrofuran (20 mL) at 0° C. under nitrogen atmosphere was added lithium aluminum hydride (4.90 mL of 2.0 M solution in tetrahydrofuran, 9.80 mmol) dropwise. The resulting reaction mixture was stirred at ambient temperature for 1.5 h, followed by the addition of sodium sulfate decahydrate. The solid was separated by filtration and washed with dichloromethane. The filtrate was concentrated and the crude material was recrystallized from ethyl acetate/hexanes to afford 1,1-(2,4,6-trimethyl-1,3-phenylene)diethanol (0.694 g, 68%): ¹H NMR (300 MHz, CDCl₃) δ 6.82 (s, 1H), 5.44 (q, J=6.6 Hz, 2H), 2.54 (d, J=5.4 Hz, 3H), 2.40 (s, 6H), 1.55 (d, J=6.6 Hz, 6H).

### Preparation 5

### Preparation of 2,5-bis(chloromethyl)thiophene

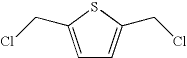

### A. Synthesis of thiophene-2,5-diyldimethanol

A solution of thiophene-2,5-dicarboxylic acid (1.40 g, 10.00 mmol) and lithium aluminium hydride (0.76 g, 20.00 mmol) in tetrahydrofuran (150 mL) was warmed up to 50° C. for 3 h, cooled to ambient temperature, neutralized with saturated sodium sulfate and filtered through celite cake. The filtrate was concentrated in vacuo and thiophene-2,5-diyldimethanol was obtained as a colorless solid 70% yield (1.01 g): MS (ES+) m/z 145.2 (M+1).

### B. Synthesis of 2,5-bis(chloromethyl)thiophene

Thiophene-2,5-diyldimethanol (1.01 g, 7.00 mmol) was dissolved in chloroform (50 mL) and 2 drops of N,N-dimethylformamide and thionyl chloride (1.67 g, 14.00 mmol) was added. The reaction mixture was stirred under nitrogen at ambient temperature for 20 h. The solvents were evaporated in vacuo and the residue was purified by column chromatography eluted with hexanes/ethyl acetate (4/1 to 1/1) to afford 2,5-bis(chloromethyl)thiophene as a colorless solid 49% yield (0.63 g): MS (ES+) m/z 182.2 (M+1).

### Preparation 6

### Preparation of 2,5-bis(bromomethyl)-3,4-diphenylthiophene

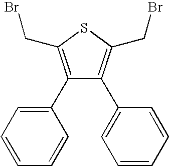

### A. Synthesis of (3,4-diphenylthiophene-2,5-diyl)dimethanol

A mixture of 3,4-diphenylthiophene-2,5-dicarboxylic acid (5.00 g, 15.00 mmol) in tetrahydrofuran (150 mL) and borane-tetrahydrofuran complex solution (22.5 mL of 2 M solution, 45 mmol) was stirred at ambient temperature for 16 h. Methanol (100 mL) was added to the mixture and followed by the addition of 10 M HCl solution (20 mL). The reaction mixture was stirred at 60° C. for 3 h and concentrated in vacuo to dryness. The residue was purified by column chromatography eluted with hexanes/ethyl acetate (2/1 to 1/1) to afford (3,4-diphenylthiophene-2,5-diyl)dimethanol as a colorless solid in 65% yield (2.90 g): MS (ES+) m/z 279.2 (M−17).

### B. Synthesis of 2,5-bis(bromomethyl)-3,4-diphenylthiophene

A mixture of (3,4-diphenylthiophene-2,5-diyl)dimethanol (2.90 g, 9.80 mmol) in dichloromethane (10 mL) and 33% hydrogen bromide solution in acetic acid (5 mL) was stirred at ambient temperature for 2 h. The reaction mixture was poured in water (100 mL) and the solid obtained was collected by filtration and dried in vacuo to afford 2,5-bis(bromomethyl)-3,4-diphenylthiophene as a colorless solid in 51% yield (2.10 g): MS (ES+) m/z 423.2 (M+1).

### Preparation 7

### Preparation of (3,4-dimethylthiophene-2,5-diyl)dimethanol

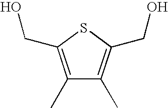

### A. Synthesis of 3,4-dimethylthiophene-2,5-dicarboxylic acid

A solution of 3,4-dimethylthiophene-2,5-dicarbonitrile (5.00 g, 31.00 mmol) and sodium hydroxide (4.00 g, 100.00 mmol) in water (50 mL) was refluxed for 24 h, cooled to ambient temperature and acidified. The solid residue was collected by filtration and dissolved in 30% sulfuric acid (100 mL). This mixture was refluxed for 20 h, cooled to ambient temperature. The solid residue was collected by filtration, washed with water and dried in vacuo to afford 3,4-dimethylthiophene-2,5-dicarboxylic acid as a colorless solid in 63% yield (3.90 g): MS (ES+) m/z 180.09 (M−17).

### B. Synthesis of dimethyl 3,4-dimethylthiophene-2,5-dicarboxylate

A mixture of 3,4-dimethylthiophene-2,5-dicarboxylic acid (3.90 g, 19.40 mmol), thionyl chloride (10.00 g, 85.00 mmol) and N,N-dimethylformamide (7.30 g, 100 mmol) in dichloromethane (50 mL) was stirred at ambient temperature for 48 h and concentrated in vacuo. The residue was refluxed in methanol (100 mL) for 16 h. The solvent was removed in vacuo and the residue was purified by column chromatography eluted with hexanes/ethyl acetate (2/1 to 1/1) to afford dimethyl 3,4-dimethylthiophene-2,5-dicarboxylate as a colorless solid in 52% yield (2.31 g): MS (ES+) m/z 229.2 (M+1).

### C. Synthesis of (3,4-dimethylthiophene-2,5-diyl)dimethanol

A mixture of dimethyl 3,4-dimethylthiophene-2,5-dicarboxylate (2.31 g, 10.00 mmol) and lithium aluminum hydride (20 mL of 2 M solution in tetrahydrofuran, 40 mmol) was stirred at ambient temperature for 24 h. The reaction mixture was neutralized with saturated sodium sulfate solution and filtered through celite. The filtrate was concentrated in vacuo and the residue was purified by column chromatography eluted with hexanes/ethyl acetate (3/1 to 1/1) to afford (3,4-dimethylthiophene-2,5-diyl)dimethanol as a colorless solid in 64% yield (1.10 g): MS (ES+) m/z 155.1 (M−17).

### Preparation 8

### Preparation of (3,4-dimethylthieno[2,3-b]thiophene-2,5-diyl)dimethanol

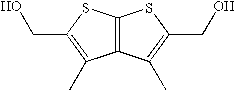

### A. Synthesis of dipropyl 3,4-dimethylthieno[2,3-b]thiophene-2,5-dicarboxylate

A solution of 3,4-dimethylthieno[2,3-b]thiophene-2,5-dicarboxylic acid (5.00 g, 19.50 mmol), thionyl chloride (10.00 g, 85.00 mmol) and N,N-dimethylformamide (7.30 g, 100.00 mmol) in dichloromethane (50 mL) was stirred at ambient temperature for 48 h. The solvents were removed in vacuo. The residue was dissolved in n-propanol (100 mL) and the resulting solution was heated under relux for 16 h. The solvent was removed in vacuo and the residue was purified by column chromatography eluted with dichloromethane/ethyl acetate (4/1 to 2/1) to afford dipropyl 3,4-dimethylthieno[2,3-b]thiophene-2,5-dicarboxylate (4.70 g, 71%) as a colorless solid: MS (ES+) m/z 341.3 (M+1).

### B. Synthesis of (3,4-dimethylthieno[2,3-b]thiophene-2,5-diyl)dimethanol

A solution of 3,4-dimethylthiophene-2,5-dicarboxylate (4.70 g, 13.8 mmol) and lithium aluminum hydride (27.5 mL of 2 M solution, 55 mmol) was stirred at ambient temperature for 48 h. After completion of the reaction, the reaction mixture was neutralized with saturated sodium sulfate solution and filtered through celite. The filtrate was concentrated in vacuo and the residue was recrystallized from toluene/hexane to afford (3,4-dimethylthieno[2,3-b]thiophene-2,5-diyl)dimethanol (2.40 g, 76%) as a colorless solid: MS (ES+) m/z 211.2 (M−17).

### Example 1

### Synthesis of N-(3-guanidinomethyl-2,4,6-trimethylbenzyl)guanidine, bis(p-toluenesulfonate)

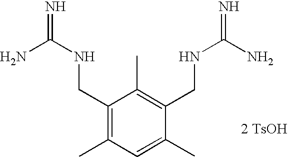

A mixture of (2,4,6-trimethyl-1,3-phenylene)dimethanamine (1.18 g, 6.62 mmol), 1-benzotriazolecarboxamidinium tosylate (prepared according to Katrizsky et al. Synth. Commun. 1995; 25(8): 1173-1186) (4.41 g, 13.2 mmol), N,N-diisopropylethylamine (2.3 mL, 13.1 mmol) and anhydrous N,N-dimethylformamide (17.0 mL) was stirred at ambient temperature for 46 h. The reaction mixture was diluted with diethyl ether (70 mL) and stirred for 10 min. The precipitate was collected by filtration, washed with diethyl ether (50 mL) and air-dried. The crude product was triturated with boiling anhydrous ethanol (50 mL) and, after cooling to ambient temperature, the solid was collected by filtration, washed with anhydrous ethanol (25 mL), air-dried and dried under high vacuum to afford N-(3-guanidinomethyl-2,4,6-trimethylbenzyl)-guanidine, bis(p-toluenesulfonate) as a colorless solid in 44% yield (1.76 g): mp>250° C. (ethanol); ¹H NMR (300 MHz, DMSO-d₆) δ 7.47-7.40 (m, 14H), 7.12 (d, J=7.2 Hz, 4H), 7.01 (s, 1H), 4.29 (d, J=4.2 Hz, 4H), 2.31-2.26 (m, 15H); ¹³C NMR (75 MHz, DMSO-d₆) δ 156.6, 145.1, 138.0, 137.4, 137.2, 130.6, 130.2, 128.2, 125.4, 20.8, 19.3, 15.0); MS (ES+) m/z 263.3 (M+1).

### Example 2

### Synthesis of 1,3-di[(2-cyano-3-methylguanidino)methyl]-2,4,6-trimethylbenzene

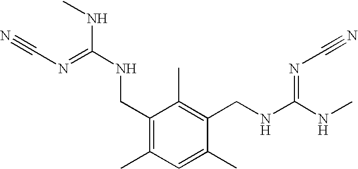

To an 8 M solution of methylamine in anhydrous ethanol (10 mL) was added 1,3-di((2-cyanoguanidino)methyl)-2,4,6-trimethylbenzene bistosylate (0.15 g, 0.40 mmol). The reaction mixture was stirred for 16 h at ambient temperature and concentrated in vacuo to dryness. The residue was recrystallized three times from boiling methanol to afford 1,3-di[(2-cyano-3-methylguanidino)methyl]-2,4,6-trimethylbenzene as a colorless solid in 5% yield (0.007 g): mp 270-272° C. (methanol); ¹H NMR (300 MHz, DMSO-d₆) δ 7.05 (m, 2H), 6.91 (s, 1H), 6.55 (br s, 2H), 4.31 (d, J=4.2 Hz, 4H), 2.68 (d, J=4.8 Hz, 6H), 2.29 (s, 6H), 2.24 (s, 3H); MS (ES+) m/z 341.6 (M+1).

### Example 3

### Synthesis of (2,4,6-trimethyl-1,3-phenylene)bis(methylene)dicarbamimidothioate dihydrochloride

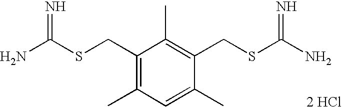

To a solution of 2,4-bis(chloromethyl)-1,3,5-trimethylbenzene (35.00 g, 161.00 mmol) in anhydrous ethanol (1000 mL) was added thiourea (24.50 g, 322.10 mmol). The reaction mixture was heated for 15 h at to 80° C. and was allowed to cool to ambient temperature, during which time a thick precipitate was deposited. The precipitate was collected by filtration, washed with ethanol (200 mL), air-dried and dried under high vacuum to afford (2,4,6-trimethyl-1,3-phenylene)bis(methylene)dicarbamimidothioate dihydrochloride as a colorless solid in 92% yield (54.0 g): mp>250° C. (ethanol); ¹H NMR (300 MHz, DMSO-d₆) δ 9.40 (br s, 8H), 7.02 (s, 1H), 4.55 (s, 4H), 2.41 (s, 3H), 2.33 (s, 6H); ¹³C NMR (75 MHz, DMSO-d₆) δ 169.9, 138.0, 137.8, 130.6, 127.7, 30.7, 19.3, 15.2; MS (ES+) m/z 297.3 (M+1).

### Example 3.1

### Synthesis of (4,6-diisopropyl-1,3-phenylene)bis(methylene)dicarbamimidothioate dihydrobromide

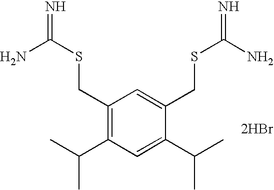

Following the procedure as described in Example 3, making non-critical variations using 1,5-bis(bromomethyl)-2,4-diisopropylbenzene to replace 2,4-bis(chloromethyl)-1,3,5-trimethylbenzene to react with thiourea, (4,6-diisopropyl-1,3-phenylene)bis(methylene)dicarbamimidothioate dihydrobromide was obtained as a white solid in 93% yield: mp 208-210° C.; ¹H NMR (300 MHz, DMSO-d₆) δ 9.34-8.87 (br s, 8H), 7.32 (s, 1H), 7.28 (s, 1H), 4.48 (s, 4H), 3.21-3.03 (m, 2H), 1.17 (d, J=6.7 Hz, 12H); ¹³C NMR (75 MHz, DMSO-d₆) δ 169.6, 148.9, 133.0, 128.4, 124.3, 32.4, 28.9, 24.4; MS (ES+) m/z 339.3 (M+1).

### Example 3.2

### Synthesis of 1,3-phenylenebis(methylene)dicarbamimidothioate dihydrobromide

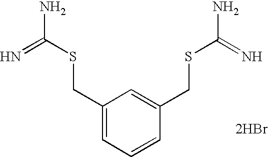

Following the procedure as described in Example 3, making non-critical variations using 1,3-bis(bromomethyl)benzene to replace 2,4-bis(chloromethyl)-1,3,5-trimethylbenzene to react with thiourea, 1,3-phenylenebis(methylene) dicarbamimidothioate was obtained as a white solid in 97% yield: mp 216-218° C.; ¹H NMR (300 MHz, DMSO-d₆) δ 9.24 (br s, 4H), 9.05 (br s, 4H), 7.46 (s, 1H), 7.39 (d, J=1.1 Hz, 3H), 4.53 (s, 4H); MS (ES+) m/z 255.4 (M+1).

### Example 3.3

### Synthesis of (5-methyl-1,3-phenylene)bis(methylene)dicarbamimidothioate dihydrobromide

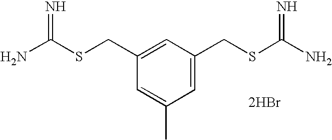

Following the procedure as described in Example 3, making non-critical variations using 1,3-bis(bromomethyl)-5-methylbenzene to replace 2,4-bis(chloromethyl)-1,3,5-trimethylbenzene to react with thiourea, (5-methyl-1,3-phenylene)bis(methylene)dicarbamimidothioate dihydrobromide was obtained as a white solid in 65% yield: mp 240-241° C.; ¹H NMR (300 MHz, CD₃OD) [symbol] 7.33 (s, 1H), 7.26 (s, 2H), 4.44 (s, 4H), 2.36 (s, 3H); ¹³CNMR (75 MHz, CD₃OD) δ 172.1, 141.2, 136.2, 131.0, 128.1, 36.1, 21.3; MS (ES+) m/z 269.5 (M+1).

### Example 3.4

### Synthesis of diethyl 4,6-bis(carbamimidoylthiomethyl)isophthalate dihydrobromide

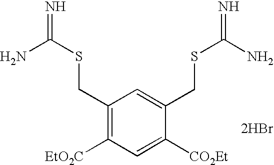

Following the procedure as described in Example 3, making non-critical variations using diethyl 4,6-bis(bromomethyl)isophthalate to replace 2,4-bis(chloromethyl)-1,3,5-trimethylbenzene to react with thiourea, diethyl 4,6-bis(carbamimidoylthiomethyl)isophthalate dihydrobromide was obtained as a white solid in 54% yield: mp 237-238° C.; ¹H NMR (300 MHz, CD₃OD) δ 8.64 (s, 1H), 7.80 (s, 1H), 4.87 (s, 4H), 4.44 (q, J=7.1 Hz, 4H), 1.43 (t, J=7.1 Hz, 6H); ¹³CNMR (75 MHz, CD₃OD) δ 172.1, 166.8, 142.2, 135.7, 135.5, 130.9, 63.3, 34.6, 14.5; MS (ES+) m/z 399.5 (M+1).

### Example 3.5

### Synthesis of (2,4,5,6-tetramethyl-1,3-phenylene)bis(methylene)dicarbamimidothioate dihydrochloride

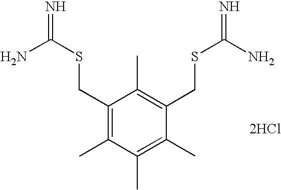

Following the procedure as described in Example 3, making non-critical variations using 1,3-bis(chloromethyl)-2,4,5,6-tetramethylbenzene to replace 2,4-bis(chloromethyl)-1,3,5-trimethylbenzene to react with thiourea, (2,4,5,6-tetramethyl-1,3-phenylene)bis(methylene)dicarbamimidothioate dihydrochloride was obtained as a white solid in 87% yield: mp>260° C.; ¹H NMR (300 MHz, DMSO-d₆) δ 9.41 (s, 8H), 4.58 (s, 4H), 2.40 (s, 3H), 2.29 (s, 6H), 2.17 (s, 3H); ¹³CNMR (75 MHz, DMSO-d₆) δ 169.8, 136.7, 134.7, 134.1, 127.1, 31.4, 16.5; MS (ES+) m/z 311.5 (M+1).

### Example 3.6

### Synthesis of (2,4,5,6-tetrachloro-1,3-phenylene)bis(methylene)dicarbamimidothioate dihydrochloride

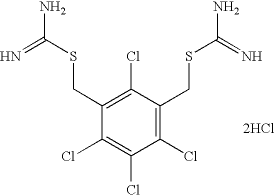

Following the procedure as described in Example 3, making non-critical variations using 1,2,3,5-tetrachloro-4,6-bis(chloromethyl)benzene to replace 2,4-bis(chloromethyl)-1,3,5-trimethylbenzene to react with thiourea, (2,4,5,6-tetrachloro-1,3-phenylene)bis(methylene)dicarbamimidothioate dihydrochloride was obtained as a white solid in 90% yield: mp 208-210° C.; ¹H NMR (300 MHz, DMSO-d₆) δ 9.57 (s, 8H), 4.79 (s, 4H); ¹³C NMR (75 MHz, DMSO-d₆) δ 168.6, 134.9, 134.8, 131.9, 131.3, 33.4; MS (ES+) m/z 393.3 (M+1).

### Example 3.7

### Synthesis of (4-amino-4H-1,2,4-triazole-3,5-diyl)bis(methylene)dicarbamimidothioate dihydrochloride

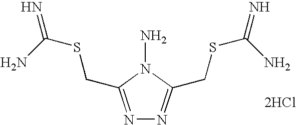

Following the procedure as described in Example 3, making non-critical variations using 3,5-bis(chloromethyl)-4H-1,2,4-triazol-4-amine (prepared according to Alonso, et al., Heterocycles 1987; 26(4): 989-1000) to replace 2,4-bis(chloromethyl)-1,3,5-trimethylbenzene to react with thiourea, (4-amino-4H-1,2,4-triazole-3,5-diyl)bis(methylene)dicarbamimidothioate dihydrochloride was obtained as a white solid in 91% yield: mp 213° C. (dec.) (ethanol); ¹H NMR (300 MHz, DMSO-d₆) δ 9.44 (br s, 4H), 9.34 (br s, 4H), 6.30 (s, 2H), 4.69 (s, 4H); ¹³C NMR (75 MHz, DMSO-d₆) δ 169.3, 151.6, 23.7; MS (ES+) m/z 261.2 (M+1).

### Example 3.8

### Synthesis of (1H-1,2,4-triazole-3,5-diyl)bis(methylene)dicarbamimidothiodate dihydrochloride

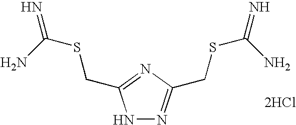

Following the procedure as described in Example 3, making non-critical variations using 3,5-bis(chloromethyl)-4H-1,2,4-triazole (Novikov, et al., Chem. Heterocycl. Compd. 1969; 5(1):121-122) to replace 2,4-bis(chloromethyl)-1,3,5-trimethylbenzene to react with thiourea, (1H-1,2,4-triazole-3,5-diyl)bis(methylene) dicarbamimidothiodate dihydrochloride was obtained as a white solid in 70% yield: mp 196-200° C. (ethanol/acetonitrile); ¹H NMR (300 MHz, DMSO-d₆) δ 9.53 (br s, 4H), 9.40 (br s, 4H), 4.64 (s, 4H); ¹³C NMR (75 MHz, DMSO-d₆) δ 169.3, 155.9, 26.2; MS (ES+) m/z 246.2 (M+1).

### Example 3.9

### Synthesis of thiophene-2,5-diylbis(methylene)dicarbamimidothioate dihydrochloride

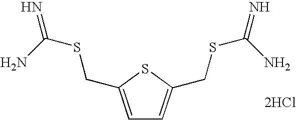

Following the procedure as described in Example 3, making non-critical variations using 2,5-bis(chloromethyl)thiophene to replace 2,4-bis(chloromethyl)-1,3,5-trimethylbenzene to react with thiourea, thiophene-2,5-diylbis(methylene) dicarbamimidothioate dihydrochloride was obtained as a white solid in 30% yield: ¹H NMR (300 MHz, DMSO-d₆) δ 9.42 (d, 8H), 6.97 (s, 2H), 4.79 (s, 4H); ¹³C NMR (75 MHz, DMSO-d₆) δ 169.2, 139.3, 128.2, 29.; MS (ES+) m/z 261.2 (M+1).

### Example 3.10

### Synthesis of (3,4-diphenylthiophene-2,5-diyl)bis(methylene)dicarbamimidothioate dihydrobromide

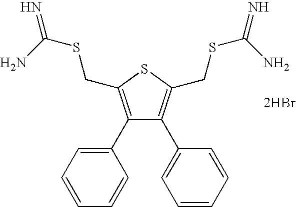

Following the procedure as described in Example 3, making non-critical variations using 2,5-bis(bromomethyl)-3,4-diphenylthiophene to replace 2,4-bis(chloromethyl)-1,3,5-trimethylbenzene to react with thiourea, (3,4-diphenylthiophene-2,5-diyl)bis(methylene)dicarbamimidothioate dihydrobromide was obtained as a white solid in 30% yield: ¹H NMR (300 MHz, DMSO-d₆) δ 9.17 (s, 4H), 9.01 (s, 4H), 7.31-6.97 (m, 10H), 4.59 (s, 4H); ¹³C NMR (75 MHz, DMSO-d₆) δ 168.9, 142.5, 134.6, 132.4, 130.2, 128.8, 128.1, 29.4; MS (ES+) m/z 413.2 (M+1).

### Example 3.11

### Synthesis of (2,4,6-trimethylbenzene-1,3,5-triyl)tris(methylene) tricarbamimidothioate trihydrobromide

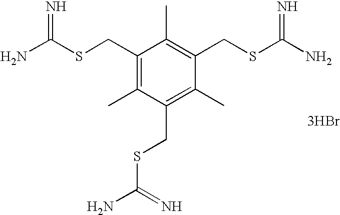

Following the procedure as described in Example 3, making non-critical variations using 1,3,5-trisbromomethyl-2,4,6-trimethylbenzene to replace 2,4-bis(chloromethyl)-1,3,5-trimethylbenzene to react with thiourea, (2,4,6-trimethylbenzene-1,3,5-triyl)tris(methylene)tricarbamimidothioate trihydrobromide was obtained as a white solid in 66% yield: mp>290 0° C. (ethanol); ¹H NMR (300 MHz, CD₃OD) δ 4.59 (s, 6H), 2.46 (s, 9H); ¹³C NMR (75 MHz, DMSO-d₆) δ 169.1, 138.3, 128.3, 31.1, 15.7; MS (ES+) m/z 385.5 (M+1).

### Example 3.12

### Synthesis of pyridine-2,6-diylbis(methylene)dicarbamimidothioate dihydrobromide

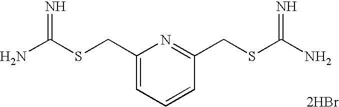

Following the procedure as described in Example 3, making non-critical variations using 2,6-bis(bromomethyl)pyridine to replace 2,4-bis(chloromethyl)-1,3,5-trimethylbenzene to react with thiourea, pyridine-2,6-diylbis(methylene) dicarbamimidothioate dihydrobromide was obtained as a white solid in 82% yield: mp 208-210° C. (ethanol); ¹H NMR (300 MHz, DMSO-d₆) δ 8.41 (s, 4H), 7.98 (s, 4H), 6.85 (t, J=7.8 Hz, 1H), 6.45 (d, J=7.8 Hz, 2H), 3.62 (s, 4H); ¹³C NMR (75 MHz, DMSO-d₆) δ 169.4, 154.8, 138.9, 122.6, 35.5; MS (ES+) m/z 256.5 (M+1).

### Example 3.13

### Synthesis of naphthalene-1,8-diylbis(methylene)dicarbamimidothioate dihydrobromide

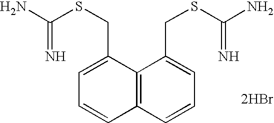

Following the procedure as described in Example 3, making non-critical variations using 1,8-bis(bromomethyl)naphthalene to replace 2,4-bis(chloromethyl)-1,3,5-trimethylbenzene to react with thiourea, naphthalene-1,8-diylbis(methylene) dicarbamimidothioate dihydrobromide was obtained as a white solid in 76% yield: mp 230-233° C. (ethanol); ¹H NMR (300 MHz, DMSO-d₆) δ 9.28 (s, 4H), 9.11 (s, 4H), 8.06 (d, J=7.9 Hz, 2H), 7.81 (d, J=7.1 Hz, 2H) 7.60-7.54 (m, 2H), 5.07 (s, 4H); MS (ES+) m/z 305.4 (M+1).

### Example 3.14

### Synthesis of (2-cyano-1,3-phenylene)bis(methylene)dicarbamimidothioate dihydrobromide

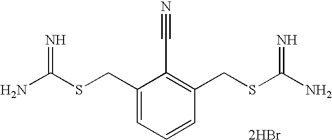

Following the procedure as described in Example 3, making non-critical variations using 2,6-bis(bromomethyl)benzonitrile to replace 2,4-bis(chloromethyl)-1,3,5-trimethylbenzene to react with thiourea, (2-cyano-1,3-phenylene)bis(methylene)dicarbamimidothioate dihydrobromide was obtained as a white solid in 90% yield: mp 270-272° C. (dec, ethanol); ¹H NMR (300 MHz, DMSO-d₆) δ 9.28 (s, 4H), 9.11 (s, 4H), 7.80-7.66 (m, 3H), 4.72 (s, 4H); ¹³C NMR (75 MHz, DMSO-d₆) δ 168.1, 139.6, 133.8, 129.9, 115.0, 112.5, 32.9; MS (ES+) m/z 280.5 (M+1).

### Example 3.15

### Synthesis of (1,2-phenylene)bis(methylene)dicarbamimidothioate dihydrobromide

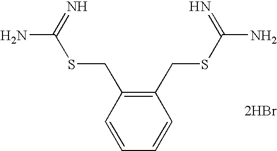

Following the procedure as described in Example 3, making non-critical variations using 1,2-bis(bromomethyl)benzene to replace 2,4-bis(chloromethyl)-1,3,5-trimethylbenzene to react with thiourea, (1,2-phenylene)bis(methylene) dicarbamimidothioate dihydrobromide was obtained as a white solid in 52% yield: mp 235-238° C. (ethanol); ¹H NMR (300 MHz, DMSO-d₆) δ 9.36 (s, 4H), 9.17 (s, 4H), 7.48-7.38 (m, 4H), 4.61 (s, 4H); ¹³C NMR (75 MHz, DMSO-d₆) δ 168.7, 133.0, 130.9, 129.0, 31.9; MS (ES+) m/z 255.5 (M+1).

### Example 3.16

### Synthesis of (4,6-dimethyl-1,3-phenylene)bis(methylene)dicarbamimidothioate dihydrochloride

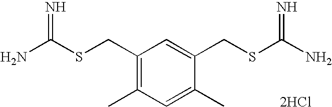

Following the procedure as described in Example 3, making non-critical variations using 1,5-bis(chloromethyl)-2,4-dimethylbenzene to replace 2,4-bis(chloromethyl)-1,3,5-trimethylbenzene to react with thiourea, (4,6-dimethyl-1,3-phenylene)bis(methylene)dicarbamimidothioate dihydrochloride was obtained as a white solid in 94% yield: mp 248-251° C. (ethanol); ¹H NMR (300 MHz, DMSO-d₆) δ 9.45 (s, 8H), 7.35 (s, 1H), 7.07 (s, 1H), 4.48 (s, 4H), 2.27 (s, 6H); ¹³C NMR (75 MHz, DMSO-d₆) δ 169.5, 137.4, 133.3, 131.6, 130.4, 32.8, 18.5; MS (ES+) m/z 283.5 (M+1).

### Example 3.17

### Synthesis of (5-bromo-4,6-dimethyl-1,3-phenylene)bis(methylene) dicarbamimidothioate dihydrochloride

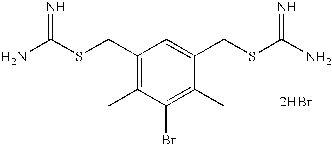

Following the procedure as described in Example 3, making non-critical variations using 3-bromo-1,5-bis(chloromethyl)-2,4-dimethylbenzene to replace 2,4-bis(chloromethyl)-1,3,5-trimethylbenzene to react with thiourea, (5-bromo-4,6-dimethyl-1,3-phenylene)bis(methylene)dicarbamimidothioate dihydrochloride was obtained as a white solid in 51% yield: mp 270-273° C. (ethanol); ¹H NMR (300 MHz, DMSO-d₆) δ 9.33 (br s, 8H), 7.44 (s, 1H), 4.58 (s, 4H), 2.45 (s, 6H); ¹³C NMR (75 MHz, DMSO-d₆) 168.8, 137.2, 131.9, 130.8, 129.8, 33.8, 20.2; MS (ES+) m/z 361.4 (M+1).

### Example 3.18

### Synthesis of (2-methoxy-5-methyl-1,3-phenylene)bis(methylene)dicarbamimidothioate dihydrobromide

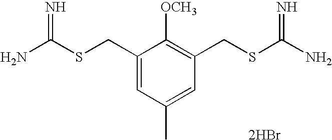

Following the procedure as described in Example 3, making non-critical variations using 1,3-bis(bromomethyl)-2-methoxy-5-methylbenzene to replace 2,4-bis(chloromethyl)-1,3,5-trimethylbenzene to react with thiourea, (2-methoxy-5-methyl-1,3-phenylene)bis(methylene)dicarbamimidothioate was obtained as a white solid in 97% yield: mp 236-239° C. (ethanol); ¹H NMR (300 MHz, DMSO-d₆) δ 9.18 (s, 4H), 9.04 (s, 4H), 7.21 (s, 2H), 4.41 (s, 4H), 3.76 (s, 3H), 2.22 (s, 3H); ¹³C NMR (75 MHz, DMSO-d₆) δ 169.2, 154.6, 134.1, 131.7, 127.8, 62.7, 29.5, 20.2; MS (ES+) m/z 299.5 (M+1).

### Example 3.19

### Synthesis of (5-hydroxy-2,4,6-trimethyl-1,3-phenylene)bis(methylene) dicarbamimidothioate dihydrochloride

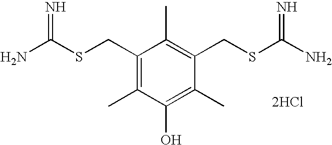

Following the procedure as described in Example 3, making non-critical variations using 3,5-bis(chloromethyl)-2,4,6-trimethylphenol to replace 2,4-bis(chloromethyl)-1,3,5-trimethylbenzene to react with thiourea, (5-hydroxy-2,4,6-trimethyl-1,3-phenylene)bis(methylene)dicarbamimidothioate dihydrochloride was obtained as a white solid in 27% yield: mp 175-178° C. (ethanol); ¹H NMR (300 MHz, CD₃OD) δ 4.53 (s, 4H), 2.43 (s, 3H), 2.33 (s, 6H); ¹³C NMR (75 MHz, DMSO-d₆) δ 171.6, 151.8, 128.8, 127.3, 126.4, 31.3, 14.1, 11.5; MS (ES+) m/z 313.6 (M+1).

### Example 3.20

### Synthesis of naphthalene-1,2-diylbis(methylene)dicarbamimidothioate dihydrobromide

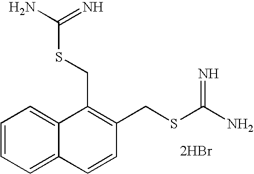

Following the procedure as described in Example 3, making non-critical variations using 1,2-bis(bromomethyl)naphthalene to replace 2,4-bis(chloromethyl)-1,3,5-trimethylbenzene to react with thiourea, naphthalene-1,2-diylbis(methylene) dicarbamimidothioate dihydrobromide was obtained as a semi solid in 89% yield: ¹H NMR (300 MHz, CD₃OD) δ 8.24-8.21 (m, 1H), 7.98-7.93 (m, 2H), 7.71-7.58 (m, 3H), 5.15 (s, 2H), 4.87 (s, 2H); ¹³C NMR (75 MHz, CD₃OD) δ 170.9, 170.5, 133.8, 131.8, 130.9, 130.3, 128.7, 127.7, 127.6, 127.2, 126.8, 123.4, 33.7, 29.1; MS (ES+) m/z 305.5 (M+1).

### Example 3.21

### Synthesis of (2-methyl-1,3-phenylene)bis(methylene)dicarbamimidothioate dihydrobromide

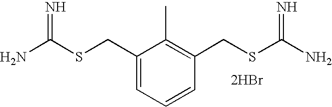

Following the procedure as described in Example 3, making non-critical variations using 1,3-bis(bromomethyl)-2-methylbenzene to replace 2,4-bis(chloromethyl)-1,3,5-trimethylbenzene to react with thiourea, (2-methyl-1,3-phenylene)bis(methylene)dicarbamimidothioate dihydrobromide was obtained as a white solid in 80% yield: mp 258-261° C. (ethanol); ¹H NMR (300 MHz, DMSO-d₆) δ 9.18-9.04 (m, 8H), 7.37-7.35 (m, 2H), 7.22-7.17 (m, 1H), 4.52 (s, 4H), 2.27 (s, 3H); ¹³C NMR (75 MHz, DMSO-d₆) δ 169.5, 137.1, 133.8, 130.9, 126.9, 33.9, 15.1; MS (ES+) m/z 269.5 (M+1).

### Example 3.22

### Synthesis of (3,4,5,6-tetramethyl-1,2-phenylene)bis(methylene)dicarbamimidothioate dihydrobromide

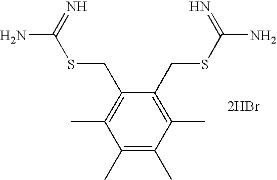

Following the procedure as described in Example 3, making non-critical variations using 1,2-bis(bromomethyl)-3,4,5,6-tetramethylbenzene to replace 2,4-bis(chloromethyl)-1,3,5-trimethylbenzene to react with thiourea, (3,4,5,6-tetramethyl-1,2-phenylene)bis(methylene)dicarbamimidothioate dihydrobromide was obtained as a semi solid in 41% yield: mp>265° C. (ethanol); ¹H NMR (300 MHz, DMSO-d₆) δ 9.42 (s, 8H), 4.65 (s, 4H), 2.29 (s, 6H), 2.19 (s, 6H); MS (ES+) m/z 311.6 (M+1).

### Example 4

### Synthesis of 2,2′-(1,3-phenylene)diacetimidamide

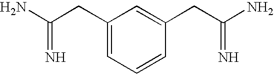

To a stirred suspension of ammonium chloride (0.69 g, 12.81 mmol) in dry toluene (3.8 mL) at 0° C. was added dropwise trimethylaluminum (2.0 M solution in toluene, 6.6 mL, 13.2 mmol). The resulting reaction mixture was stirred at ambient temperature for 1.5 h and 1,3-phenylenediacetonitrile (0.50 g, 3.20 mmol) in dry toluene (2.1 mL) was added at ambient temperature. The resulting reaction mixture was stirred at reflux for 5 h, cooled to ambient temperature and poured into slurry of silica gel (20 g) in dichloromethane (20 mL) and the mixture was stirred for 5 minutes. The silica gel was separated by filtration and washed with methanol (100 mL). The filtrate was concentrated in vacuo and the residue was purified by LC/MS and the fractions were collected and dried in vacuo to afford 2,2′-(1,3-phenylene)diacetimidamide as a white waxy solid (0.06 g): mp. 200-205° C.; ¹H NMR (300 MHz, CD₃OD) δ 7.52-7.35 (m, 4H), 3.86 (s, 4H); ¹³C NMR (75 MHz, CD₃OD) δ 171.3, 135.5, 131.0, 130.98, 129.83, 39.1; MS (ES+) m/z 191.3 (M+1).

### Example 5

### Synthesis of 1,3-phenylene dicarbamimidothioate dihydroiodide

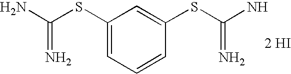

A flask containing 1,3-diiodobenzene (0.50 g, 1.52 mmol), bis(triethylphosphine)nickel(II) chloride (0.028 g, 0.050 mmol), sodium cyanoborohydride (0.007 g, 0.072 mmol) and thiourea (0.35 g, 4.60 mmol) was flushed with nitrogen. Anhydrous N,N-dimethylformamide (3 mL) was added and the flask was again flushed with nitrogen. The reaction mixture was stirred at 80° C. for 4 h, allowed to cool to ambient temperature, diluted with water (25 mL) and extracted with dichloromethane (3×25 mL). The aqueous layer was concentrated and the residue was heated at reflux in ethanol (10 mL) for 15 minutes. The solution was filtered while hot and the filtrate was allowed to cool to ambient temperature, and then concentrated. The residue was purified by column chromatography and dried in vacuo to afford 1,3-phenylene dicarbamimidothioate dihydroiodide as a brown oil: ¹H NMR (300 MHz, CD₃OD) δ 8.17 (dd, J=1.6, 1.6 Hz, 1H), 8.00 (dd, J=1.6, 7.9 Hz, 2H), 7.84-7.76 (m, 1H); ¹³CNMR (75 MHz, CD₃OD) δ 170.1, 144.2, 133.2, 125.5; MS (ES+) m/z 227.3 (M+1).

### Example 6

### Synthesis of 2-{1-[3-(1-carbamimidoylsulfanyl-1-methylethyl)phenyl]-1-methylethyl}-isothiourea dihydrobromide

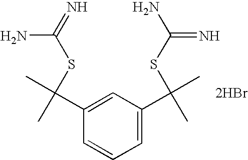

To a stirred suspension of thiourea (0.39 g, 5.15 mmol) in 48% aqueous hydrobromic acid (2 mL) was added 2,2′-(1,3-phenylene)dipropan-2-ol (0.50 g, 2.57 mmol) at 0° C. The resulting thick paste was stirred at 0° C. for 2 h and ice-cold water (15 mL) was added. The white precipitate was collected by filtration and washed with ether. The solid was recrystallized from hot ethanol/ether to afford 2-{1-[3-(1-carbamimidoylsulfanyl-1-methylethyl)phenyl]-1-methylethyl}isothiourea dihydrobromide as white crystals in 17% yield (0.21 g): mp 142-144° C.; ¹H NMR (300 MHz, CD₃OD) δ 7.91-7.88 (m, 1H), 7.71-7.66 (m, 2H), 7.58-7.52 (m, 1H), 1.99 (s, 12H); ¹³C NMR (75 MHz, CD₃OD) δ 169.3, 145.4, 131.1, 127.7, 125.8, 56.9, 31.2; MS (ES+) m/z 311.5 (M+1).

### Example 6.1

### Synthesis of 2-{1-[3-(1-carbamimidoylsulfanylethyl)-2,4,6-trimethylphenyl]ethyl}-isothiourea dihydrobromide

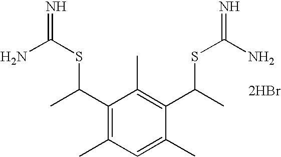

Following the procedure as described in Example 6, making non-critical variations using 1,1-(2,4,6-trimethyl-1,3-phenylene)diethanol to replace 2,2′-(1,3-phenylene)dipropan-2-ol, 2-{1-[3-(1-carbamimidoylsulfanyl-ethyl)-2,4,6-trimethyl-phenyl]-ethyl}-isothiourea dihydrobromide was obtained as a white solid in 57% yield: mp 204-206° C.; ¹H NMR (300 MHz, DMSO-d₆) δ 9.34 (s, 4H), 9.11 (s, 4H), 6.96 (s, 1H), 5.52-5.24 (m, 2H), 2.5 (s, 3H), 2.44 (s, 3H), 2.34 (s, 3H), 1.74 (d, J=6.6 Hz, 6H); MS (ES+) m/z 325.6 (M+1).

### Example 6.2

### Synthesis of (2-hydroxy-5-methyl-1,3-phenylene)bis(methylene)dicarbamimidothioate dihydrobromide

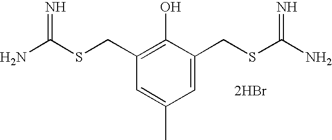

Following the procedure as described in Example 6, making non-critical variations using 2,6-bis(hydroxymethyl)-p-cresol to replace 2,2′-(1,3-phenylene)dipropan-2-ol, of (2-hydroxy-5-methyl-1,3-phenylene)bis(methylene)dicarbamimidothioate dihydrobromide was obtained as a white solid in 20% yield: mp 223-225° C.; ¹H NMR (300 MHz, DMSO-d₆) δ 9.36 (s, 1H), 9.14 (s, 4H), 9.01 (s, 4H), 7.11 (s, 2H), 4.42 (s, 4H), 2.17 (s, 3H); ¹³CNMR (75 MHz, DMSO-d₆) δ 169.6, 151.2, 131.5, 128.7, 121.8, 30.6, 19.9; MS (ES+) m/z 285.5 (M+1).

### Example 6.3

### Synthesis of (3,4-dimethylthiophene-2,5-diyl)bis(methylene)dicarbamimidothioate dihydrobromide

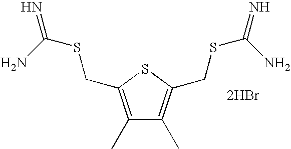

Following the procedure as described in Example 6, making non-critical variations using (3,4-dimethylthiophene-2,5-diyl)dimethanol to replace 2,2′-(1,3-phenylene)dipropan-2-ol, the title compound was obtained as a white solid in 73% yield: ¹H NMR (300 MHz, DMSO-d₆) δ 9.23 (s, 4H), 9.06 (s, 4H), 4.70 (s, 4H), 2.03 (s, 6H); ¹³C NMR (75 MHz, DMSO-d₆) δ 169.1, 137.7, 129.3, 28.9, 13.1; MS (ES+) m/z 289.2 (M+1).

### Example 6.4

### Synthesis of (3,4-dimethylthieno[2,3-b]thiophene-2,5-diyl)bis(methylene)dicarbamimidothioate dihydrobromide

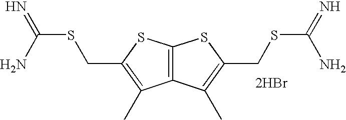

Following the procedure as described in Example 6, making non-critical variations using (3,4-dimethylthieno[2,3-b]thiophene-2,5-diyl)dimethanol to replace 2,2′-(1,3-phenylene)dipropan-2-ol, (3,4-dimethylthieno[2,3-b]thiophene-2,5-diyl)bis(methylene)dicarbamimidothioate dihydrobromide was obtained as a white solid in 99% yield: ¹H NMR (300 MHz, DMSO-d₆) δ 9.26 (s, 4H), 9.08 (s, 4H), 4.80 (s, 4H), 2.39 (s, 6H); ¹³C NMR (75 MHz, DMSO-d₆) δ 169.0, 146.3, 134.9, 132.6, 130.8, 29.6, 13.2; MS (ES+) m/z 345.4 (M+1).

### Example 6.5

### Synthesis of (4-methoxy-1,3-phenylene)bis(methylene)dicarbamimidothioate dihydrobromide

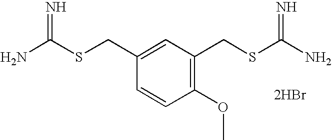

Following the procedure as described in Example 6, making non-critical variations using (4-methoxy-1,3-phenylene)dimethanol to replace 2,2′-(1,3-phenylene)dipropan-2-ol, (4-methoxy-1,3-phenylene)bis(methylene)dicarbamimidothioate dihydrobromide was obtained as a white solid in 94% yield: ¹H NMR (300 MHz, CDCl₃) δ 9.16 (s, 4H), 8.99 (m, 4H), 7.39 (s, 1H), 7.37 (dd, J=8.5, 2.1 Hz, 1H), 7.03 (d, 1H, J=8.5 Hz), 4.42 (s, 2H), 4.36 (s, 2H), 3.79 (s, 3H); MS (ES+) m/z 285.5 (M+1).

### Example 6.6

### Synthesis of (5-methoxy-1,3-phenylene)bis(methylene)dicarbamimidothioate dihydrobromide

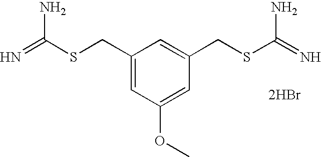

Following the procedure as described in Example 6, making non-critical variations using (5-methoxy-1,3-phenylene)dimethanol to replace 2,2′-(1,3-phenylene)dipropan-2-ol, (5-methoxy-1,3-phenylene)bis(methylene)dicarbamimidothioate dihydrobromide was obtained as a white solid in 95% yield: ¹H NMR (300 MHz, CDCl₃) δ 9.16 (s, 4H), 8.99 (s, 4H), 6.99-6.96 (m, 1H), 6.96-6.93 (m, 2H), 4.43 (s, 4H), 3.72 (s, 3H); MS (ES+) m/z 285.5 (M+1).

### Example 7

### Synthesis of 1,3-di[(methylamidino)thiomethyl]-2,4,6-trimethylbenzene 4-methylbenzenesulfonate hydrochloride

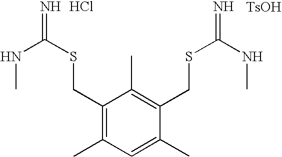

A mixture of 2,4-bis(chloromethyl)-1,3,5-trimethylbenzene (0.50 g, 2.30 mmol) and 1-methyl-2-thiourea (0.42 g, 4.60 mmol) in absolute ethanol (10 mL) was refluxed for 16 h and cooled to ambient temperature. To the reaction mixture was added 2.0 M ammonia in methanol (2.5 mL, 5.00 mmol) dropwise at 0° C., stirred for 30 min and filtered. p-Toluenesulfonic acid monohydrate (0.95 g, 5.01 mmol) was added to the filtrate, and the resulting mixture was stirred for 30 minutes and concentrated to one half of the original volume and acetonitrile was added. The white solid obtained was collected by filtration, washed with acetonitrlie and dried in vacuo to afford 1,3-di[(methylamidino)thiomethyl]-2,4,6-trimethylbenzene 4-methylbenzenesulfonate hydrochloride as a white solid in 68% yield (0.84 g): mp 223-225° C.; ¹H NMR (300 MHz, DMSO-d₆) δ 9.88 (d, J=4.7 Hz, 2H), 9.53 (s, 2H), 9.23 (s, 2H), 7.47 (d, J=8.0 Hz, 2H), 7.10 (d, J=8.0 Hz, 2H), 7.00 (s, 1H), 4.57 (s, 4H), 2.94 (d, J=4.7 Hz, 6H), 2.41 (s, 3H), 2.33 (s, 6H), 2.27 (s, 3H); ¹³C NMR (75 MHz, DMSO-d₆) δ 166.0, 145.4, 138.0, 137.8, 137.6, 130.5, 128.0, 127.8, 125.4, 31.1, 30.6, 20.7, 19.3, 15.1; MS (ES+) m/z 324.5 (M+1).

### Example 8

### Synthesis of (2,4,6-trimethylpyridine-3,5-diyl)bis(methylene)dicarbamimidothioate dihydrochloride

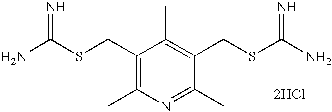

A mixture of (5-hydroxymethyl-2,4,6-trimethylpyridin-3-yl)methanol (0.10 g, 0.55 mmol) and thionylchloride (5 mL) was refluxed for 10 min and then concentrated to dryness in vacuo. The residue and thiourea (0.08 g, 1.10 mmol) were dissolved in anhydrous ethanol (50 mL). The resulting mixture was refluxed for 4 h, cooled to room temperature and concentrated in vacuo. The residue was dissolved in minimum amount of methanol (2-3 mL) and triturated with acetonitrile. The white solid obtained was collected by filtration, washed with acetonitrile, and dried in vacuo. (2,4,6-trimethylpyridine-3,5-diyl)bis(methylene)dicarbamimidothioate dihydrochloride was obtained as a white crystals in 14% yield (0.02 g): mp 185-187° C. (ethanol); ¹H NMR (300 MHz, CD₃OD) δ 4.47 (s, 4H), 2.87 (s, 6H), 2.79 (s, 3H); ¹³C NMR (75 MHz, DMSO-d₆) δ 171.2, 160.5, 154.2, 130.1, 30.6, 18.4, 17.6; MS (ES+) m/z 298.5 (M+1)

### Example 9

### Synthesis of 2-(5-carbamimidoylsulfanecarbonyl-3,4-dichlorothiophene-2-carbonyl)isothiourea dihydrochloride

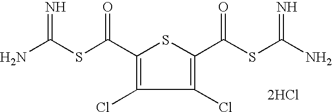

A mixture of 3,4-dichlorothiophene-2,5-dicarbonyl dichloride (0.07 g, 0.25 mmol) and thiourea (0.04 g, 0.49 mmol) was refluxed in benzene (5 mL) for 1 h and cooled to ambient temperature. The mixture was concentrated in vacuo and the residue was recrystallized from ethanol to afford 2-(5-carbamimidoylsulfanecarbonyl-3,4-dichlorothiophene-2-carbonyl)isothiourea dihydrochloride as a white solid in 95% yield (0.08 g): mp 215-218° C. (ethanol); ¹H NMR (300 MHz, DMSO-d₆) δ 7.01 (m, 8H); ¹³C NMR (75 MHz, DMSO-d₆) δ 184.3, 160.7, 131.1, 129.3.

### Example 10

In a similar manner as described above utilizing the appropriately substituted starting materials, the following compounds of the invention were prepared: (2-fluoro-1,3-phenylene)bis(methylene)dicarbamimidothioate; and (4,6-dibromo-1,3-phenylene)bis(methylene)dicarbamimidothioate; mP 161-163° C.; ¹H NMR (300 MHz, CD₃OD) δ 6.44 (s, 1H), 6.18 (s, 1H), 2.97 (s, 4H); ¹³C NMR (75 MHz, CD₃OD) δ 168.7, 135.6, 132.3, 131.7, 123.5, 33.6; MS (ES+) m/z 411.0 (M+1), 413.0 (M+1), 415.0 (M+1).

### BIOLOGICAL ASSAYS

Various techniques are known in the art for testing the activity of compounds of the invention. In order that the invention described herein may be more fully understood, the following biological assays are set forth. It should be understood that these examples are for illustrative purposes only and are not to be construed as limiting this invention in any manner.

### Biological Example 1

### DMT1 Activity Assay (In Vitro Assay)

This example discloses various in vitro assay for testing and profiling test agents against DMT1 stably expressed in cells of either an endogenous or recombinant origin. These assays can use stable cell lines overexpressing DMT1 or intestinal cells and intestinal tissue expressing endogenous DMT1. DMT1 function could also be assessed in other cell types that express DMT1. Of greatest relevance would be the erythrocytes (e.g. K562 cells) or hepatocytes (e.g. HepG3).

DMT1 function can be assessed in a number of ways, including monitoring fluorescence changes of an iron fluorophore (e.g. calcein), monitoring uptake of radiolabelled iron (⁵⁵Fe or ⁵⁹Fe) (Picard et al., J. Biol. Chem., 2000, 275(46):35738-45 and Wetli et al., Chem. Biol. 2006 September; 13(9):965-72), or by assessing the current or transport of iron and other metals into the cells or tissues using standard electrophysiological techniques (Gunshin et al., Nature, 1997, 388(6641):482-8.).

Variations of these assays involve alterations of incubation times, the iron status of the cells and tissues (which may be modulated by chemical chelators or by harvesting from iron deficient animals), the metal cation detected and the pH of the reaction can generally be made by conventional techniques known to those skilled in the art.

### Biological Example 2

### In Vivo Assay for Treatment of Iron Disorders

This test measures the efficacy of compounds of the invention in blocking ferrous iron uptake in the duodenum in rats. The animals were rendered iron deficient by feeding an iron deficient diet for 3 weeks, which causes a marked decrease in serum iron and transferrin saturation. As a result of the iron deficiency, DMT1 expression in the duodenum is upregulated. The test animals were then given an oral bolus (or an “iron challenge”) of ferrous iron at 1 mg/Kg resulting in a 20-fold increase in serum iron 1 hour post challenge. It was observed that when test animals were dosed with compound 1 hour prior to the iron challenge, there was a substantial reduction in the increase in serum iron level 1 hour post iron challenge. Compounds of the present invention were shown to be efficacious within a range of 30 mg/Kg and 0.1 mg/Kg.

Representative compounds of the invention, when tested in the above assay, demonstrated an IC₅₀ (nM) activity level as set forth below in Table 1 wherein “A” refers to an IC₅₀ activity level of from 1 nM to 10 nM, “B” refers to an IC₅₀ activity level from 10 nM to 100 nM, “C” refers to an IC₅₀ activity level from 100 nM to 1.0 μM, and “D” refers to an IC₅₀ activity level equal to or greater than 1.0 μM. The Example numbers provided in Table 1 correspond to the Examples herein:

TABLE 1

| Example | | IC₅₀ Activity |
|---|---|---|
| No. | Compound Name | Level |
| 1 | N-(3-guanidinomethyl-2,4,6-trimethylbenzyl)guanidine, | D |
| | bis(p-toluenesulfonate) | |
| 2 | 1,3-di[(2-cyano-3-methylguanidino)methyl]-2,4,6- | D |
| | trimethylbenzene | |
| 3 | (2,4,6-trimethy1-1,3- | C |
| | phenylene)bis(methylene)dicarbamimidothioate | |
| | dihydrochloride | |
| 3.1 | (4,6-diisopropyl-1,3-phenylene)bis(methylene) | D |
| | dicarbamimidothioate dihydrobromide | |
| 3.2 | 1,3-phenylenebis(methylene) dicarbamimidothioate | D |
| 3.3 | (5-methyl-1,3-phenylene)bis(methylene) | D |
| | dicarbamimidothioate | |
| 3.4 | diethyl 4,6-bis(carbamimidoylthiomethyl)isophthalate | D |
| 3.5 | (2,4,5,6-tetramethyl-1,3-phenylene)bis(methylene) | D |
| | dicarbamimidothioate | |
| 3.6 | (2,4,5,6-tetrachloro-1,3-phenylene)bis(methylene) | D |
| | dicarbamimidothioate | |
| 3.7 | (4-amino-4H-1,2,4-triazole-3,5-diyl)bis(methylene) | D |
| | dicarbamimidothioate | |
| 3.8 | (1H-1,2,4-triazole-3,5-diyl)bis(methylene) | D |
| | dicarbamimidothiodate | |
| 3.9 | thiophene-2,5-diylbis(methylene) dicarbamimidothioate | D |
| 3.10 | (3,4-diphenylthiophene-2,5-diyl)bis(methylene) | D |
| | dicarbamimidothioate | |
| 3.11 | (2,4,6-trimethylbenzene-1,3,5-triyl)tris(methylene) | D |
| | tricarbamimidothioate | |
| 3.12 | pyridine-2,6-diylbis(methylene) dicarbamimidothioate | D |
| 3.13 | naphthalene-1,8-diylbis(methylene) dicarbamimidothioate | D |
| 3.14 | (2-cyano-1,3-phenylene)bis(methylene) | D |
| | dicarbamimidothioate | |
| 3.15 | (1,2-phenylene)bis(methylene) dicarbamimidothioate | D |
| 3.16 | (4,6-dimethyl-1,3-phenylene)bis(methylene) | C |
| | dicarbamimidothioate | |
| 3.17 | (5-bromo-4,6-dimethyl-1,3-phenylene)bis(methylene) | D |
| | dicarbamimidothioate | |
| 3.18 | (2-methoxy-5-methyl-1,3-phenylene)bis(methylene) | D |
| | dicarbamimidothioate | |
| 3.19 | (5-hydroxy-2,4,6-trimethyl-1,3-phenylene)bis(methylene) | C |
| | dicarbamimidothioate | |
| 3.20 | naphthalene-1,2-diylbis(methylene) dicarbamimidothioate | D |
| 3.21 | (2-methyl-1,3-Phenylene)bis(methylene) | C |
| | dicarbamimidothioate | |
| 3.22 | (3,4,5,6-tetramethyl-1,2-phenylene)bis(methylene) | D |
| | dicarbamimidothioate | |
| 4 | 2,2′-(1,3-phenylene)diacetimidamide | D |
| 5 | 1,3-phenylene dicarbamimidothioate | D |
| 6 | 2-{1-[3-(1-carbamimidoylsulfanyl-1-methylethyl)phenyl]-1- | D |
| | methylethyl}isothiourea | |
| 6.1 | 2-{1-[3-(1-carbamimidoylsulfanylethyl)-2,4,6-trimethyl- | C |
| | phenyl]ethyl}isothiourea | |
| 6.2 | (2-hydroxy-5-methyl-1,3-phenylene)bis(methylene) | D |
| | dicarbamimidothioate | |
| 6.3 | (3,4-dimethylthiophene-2,5-diyl)bis(methylene) | D |
| | dicarbamimidothioate | |
| 6.4 | (3,4-dimethylthieno[2,3-b]thiophene-2,5-diyl)bis(methylene) | D |
| | dicarbamimidothioate | |
| 6.5 | (4-methoxy-1,3-phenylene)bis(methylene) | D |
| | dicarbamimidothioate | |
| 6.6 | (5-methoxy-1,3-phenylene)bis(methylene) | D |
| | dicarbamimidothioate | |
| 7 | 1,3-di[(methylamidino)thiomethyl]-2,4,6-trimethylbenzene 4- | D |
| | methylbenzenesulfonate | |
| 8 | (2,4,6-trimethylpyridine-3,5-diyl)bis(methylene) | D |
| | dicarbamimidothioate | |
| 9 | 2-(5-carbamimidoylsulfanecarbonyl-3,4-dichlorothiophene-2- | D |
| | carbonyl)isothiourea | |
| 10 | (2-fluoro-1,3-phenylene)bis(methylene) | D |
| | dicarbamimidothioate | |
| 10 | (4,6-dibromo-1,3-phenylene)bis(methylene) | C |
| | dicarbamimidothioate | |

A variation of this assay can be used for longer term studies. In this variation, animals are again rendered iron deficient by feeding of an iron deficient diet for 3 weeks. Then animals are switched back to an iron replete diet, while receiving a daily dose of either vehicle or a compound described herein. The vehicle animals recover their iron status, as measured by serum iron and other iron indicies, after 13 days. The drug treated animals, however, do not recover in this timeframe, as the compound is blocking the uptake of dietary iron. Other parameters that can be measured in both models include transferrin saturation, haemoglobin, hematocrit, liver iron and ferritin. More detailed assays can involve the use of radioactive metals as opposed to a bolus of ferrous iron. Multiple metals transported by DMT1 can be used to judge specificity of compound on cation uptake by DMT1, if any.

Genetic rat models of iron overload offers another format to show efficacy of DMT1 inhibitors in preventing further iron loading as development proceeds. These models are applicable to variety of human iron overload disorders such as hereditary hemochromatosis (Levy et al, Blood, 1999, 94:9-11, 1999), juvenile hemochromatosis (Huang et al, J. Clin. Invest., 2005 115:2187-2191), beta-2-microglobulin (de Sousa et al., Immun. Lett., 1994, 39:105-111, 1994), thalassemia (Ciavatta et al., Proc. Nat. Acad. Sci., 1995, 92: 9259-9263), hypotransferrinmia (Craven et. al., Proc. Nat. Acad. Sci., 1987, USA. 84(10):3457-61) and other hypochromic microcytic anemias.

In these models, the knock-out animals above are bred and treated with compound as they develop. Compound efficacy can be assessed by measuring reduced iron flux via the duodenum in a radioactive flux study or by monitoring whether chronic exposure to compounds cause a decrease in the amount of iron loading, as judged by serum iron, transferrin saturation, ferritin and liver iron. These models can be used with an iron bolus, or challenge, as above or iron may be absorbed from the diet. Where appropriate, a model of transfusional iron overload can be created in the rodent by transfusion of iron from another animal in order to exacerbate the iron overload is as seen clinically in the treatment of thalassemia.

All of the U.S. patents, U.S. patent application publications, U.S. patent applications, foreign patents, foreign patent applications and non-patent publications referred to in this specification are incorporated herein by reference in their entireties. Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

What is claimed is:

1. A method of treating a disease or condition in a mammal wherein the disease or condition is selected from the group consisting of iron overload, transfusional iron overload and thalassemia and wherein the method comprises administering to the mammal a therapeutically effective amount of a compound of formula (I): 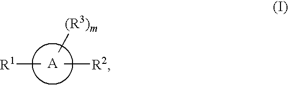
wherein:
m is 0, 1, 2, 3, or 4; 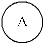
is aryl or heteroaryl;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R³ is independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl;
as a stereoisomer, enantiomer, tautomer thereof or mixtures thereof;
or a pharmaceutically acceptable salt, solvate or prodrug thereof.

2. The method of claim 1 wherein the compound of formula (I) is a compound of formula (Ia): 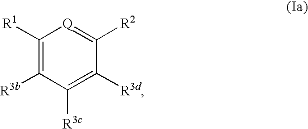
wherein:
Q is —C(R^{3a})═ or —N═;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

3. The method of claim 2 wherein the compound of formula (I) is a compound of formula (Ia) wherein:
Q is —C(R^{3a})═;
R¹ and R² are the same and are selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

4. The method of claim 3 wherein the compound of formula (I) is a compound of formula (Ia) wherein:
Q is —C(R^{3a})═;
R¹ and R² are each —R⁶—S—C(═NR⁴)N(R⁴)R⁵;
R^{3a}, R^{3b}, R^{3c} and R^{3d} are each independently selected from the group consisting of hydrogen, alkyl, halo, —R⁶—OR⁷, —R⁶—CN, —R⁶—C(O)OR⁸ and —R⁶—S—C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

5. The method of claim 4 wherein the compound of formula (I) is a compound of formula (Ia) selected from the group consisting of:
1,3-phenylenebis(methylene)dicarbamimidothioate;
(2,4,6-trimethyl-1,3-phenylene)bis(methylene)dicarbamimidothioate;
(2-fluoro-1,3-phenylene)bis(methylene)dicarbamimidothioate;
1,3-phenylene dicarbamimidothioate;
(5-methyl-1,3-phenylene)bis(methylene)dicarbamimidothioate;
(2,4,6-trimethylbenzene-1,3,5-triyl)tris(methylene) tricarbamimidothioate;
2-{1-[3-(1-carbamimidoylsulfanyl-1-methylethyl)phenyl]-1-methylethyl}isothiourea;
(2-cyano-1,3-phenylene)bis(methylene)dicarbamimidothioate;
(4,6-dimethyl-1,3-phenylene)bis(methylene)dicarbamimidothioate;
diethyl 4,6-bis(carbamimidoylthiomethyl)isophthalate;
(5-bromo-4,6-dimethyl-1,3-phenylene)bis(methylene)dicarbamimidothioate;
(2,4,5,6-tetramethyl-1,3-phenylene)bis(methylene)dicarbamimidothioate;
2-{1-[3-(1-carbamimidoylsulfanylethyl)-2,4,6-trimethylphenyl]ethyl}isothiourea;
(2-hydroxy-5-methyl-1,3-phenylene)bis(methylene)dicarbamimidothioate;
1,3-di[(methylamidino)thiomethyl]-2,4,6-trimethylbenzene;
(5-hydroxy-2,4,6-trimethyl-1,3-phenylene)bis(methylene)dicarbamimidothioate;
(2,4,5,6-tetrachloro-1,3-phenylene)bis(methylene)dicarbamimidothioate;
(2-methoxy-5-methyl-1,3-phenylene)bis(methylene)dicarbamimidothioate;
(2-methyl-1,3-phenylene)bis(methylene)dicarbamimidothioate;
(4-methoxy-1,3-phenylene)bis(methylene)dicarbamimidothioate;
(5-methoxy-1,3-phenylene)bis(methylene)dicarbamimidothioate;
(4,6-dibromo-1,3-phenylene)bis(methylene)dicarbamimidothioate; and
(4,6-diisopropyl-1,3-phenylene)bis(methylene)dicarbamimidothioate.

6. The method of claim 3 wherein the compound of formula (I) is a compound of formula (Ia) wherein:
Q is —C(R^{3a})═;
R¹ and R² are the same and selected from —R⁶—N(R⁷)C(═NCN)N(R⁴)R⁵ and R⁶—C(═NR⁴)N(R⁴)R⁵;
R^{3a}, R^{3b}, R^{3c} and R^{3d} are each independently selected from the group consisting of hydrogen, alkyl, halo, —R⁶—OR⁷, —R⁶—CN, —R⁶—C(O)OR⁸ and —R⁶—S—C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

7. The method of claim 6 wherein the compound of formula (Ia) is selected from the group consisting of:
1,3-di[(2-cyano-3-methylguanidino)methyl]-2,4,6-trimethylbenzene; and
2,2′-(1,3-phenylene)diacetimidamide.

8. The method of claim 3 wherein the compound of formula (I) is a compound of formula (Ia) wherein:
Q is —C(R^{3a})═;
R¹ and R² are each —R⁶—N(R⁷)C(═NR⁴)N(R⁴)R⁵;
R^{3a}, R^{3b}, R^{3c} and R^{3d} are each independently selected from the group consisting of hydrogen, alkyl, halo, —R⁶—OR⁷, —R⁶—CN, —R⁶—C(O)OR⁸ and —R⁶—S—C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

9. The method of claim 8 wherein the compound of formula (Ia) is N-(3-guanidinomethyl-2,4,6-trimethylbenzyl)guanidine.

10. The method of claim 2 wherein the compound of formula (I) is a compound of formula (Ia) wherein:
Q is —N═;
R¹ and R² are the same and are selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

11. The method of claim 10 wherein the compound of formula (I) is a compound of formula (Ia) wherein:
Q is —N═;
R¹ and R² are each —R⁶—S—C(═NR⁴)N(R⁴)R⁵;
R^{3b}, R^{3c} and R^{3d} are each independently selected from the group consisting of hydrogen, alkyl, and halo;
each R⁴ and R⁵ is independently hydrogen, alkyl, or —OR⁷;
each R⁶ is independently a direct bond or a straight or branched alkylene chain; and
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl.

12. The method of claim 11 wherein the compound of formula (Ia) is selected from the group consisting of:
pyridine-2,6-diylbis(methylene)dicarbamimidothioate; and
(2,4,6-trimethylpyridine-3,5-diyl)bis(methylene)dicarbamimidothioate.

13. The method of claim 1 wherein the compound of formula (I) is a compound of formula (Ib): 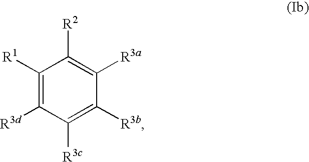
wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

14. The method of claim 13 wherein the compound of formula (I) is a compound of formula (Ib) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

15. The method of claim 14 wherein the compound of formula (I) is a compound of formula (Ib) wherein:
R¹ and R² are the same and are —R⁶—S—C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

16. The method of claim 15 wherein the compound of formula (Ib) is selected from the group consisting of:
(1,2-phenylene)bis(methylene)dicarbamimidothioate; and
(3,4,5,6-tetramethyl-1,2-phenylene)bis(methylene)dicarbamimidothioate.

17. The method of claim 1 wherein the compound of formula (I) is a compound of formula (Ic): 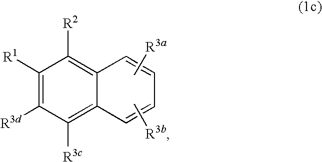
wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

18. The method of claim 17 wherein the compound of formula (I) is a compound of formula (Ic) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

19. The method of claim 18 wherein the compound of formula (I) is a compound of formula (Ic) wherein:
R¹ and R² are both —R⁶—S—C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

20. The method of claim 19 wherein the compound of formula (Ic) is naphthalene-1,2-diylbis(methylene)dicarbamimidothioate.

21. The method of claim 1 wherein the compound of formula (I) is a compound of formula (Id): 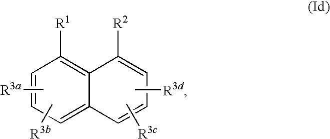
wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

22. The method of claim 21 wherein the compound of formula (I) is a compound of formula (Id) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

23. The method of claim 22 wherein the compound of formula (I) is a compound of formula (Id) wherein:
R¹ and R² are both —R⁶—S—C(═NR⁴)N(R⁴)R⁵;
R^{3a}, R^{3b}, R^{3c} and R^{3d} are each independently selected from the group consisting of hydrogen, alkyl, halo, haloalkyl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

24. The method of claim 23 wherein the compound of formula (Id) is naphthalene-1,8-diylbis(methylene)dicarbamimidothioate.

25. The method of claim 1 wherein the compound of formula (I) is a compound of formula (Ie): 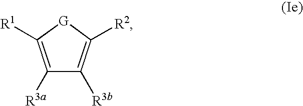
wherein:
G is —O— or —S—;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

26. The method of claim 25 wherein the compound of formula (I) is a compound of formula (Ie) wherein:
G is —O— or —S—;
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

27. The method of claim 26 wherein the compound of formula (I) is a compound of formula (Ie) wherein:
G is —S—;
R¹ and R² are the same and selected from —R⁶—S—C(═NR⁴)N(R⁴)R⁵ and —R⁶—C(O)—S—C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

28. The method of claim 27 wherein the compound of formula (Ie) is selected from the group consisting of:
2-(5-carbamimidoylsulfanecarbonyl-3,4-dichlorothiophene-2-carbonyl)isothiourea;
thiophene-2,5-diylbis(methylene)dicarbamimidothioate;
(3,4-diphenylthiophene-2,5-diyl)bis(methylene)dicarbamimidothioate; and
(3,4-dimethylthiophene-2,5-diyl)bis(methylene)dicarbamimidothioate.

29. The method of claim 1 wherein the compound of formula (I) is a compound of formula (If): 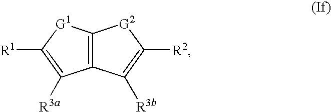
wherein:
G¹ and G² are both —O—;
or G¹ and G² are both —S—;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

30. The method of claim 29 wherein the compound of formula (I) is a compound of formula (If) wherein:
G¹ and G² are both —O—;
or G¹ and G² are both —S—;
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl.

31. The method of claim 30 wherein the compound of formula (I) is a compound of formula (If) wherein:
G¹ and G² are both —S—;
R¹ and R² are the same and selected from —R⁶—S—C(═NR⁴)N(R⁴)R⁵ and —R⁶—C(O)—S—C(═NR⁴)N(R⁴)R⁵;
R^{3a} and R^{3b} are each independently selected from the group consisting of hydrogen, alkyl, halo, haloalkyl, optionally substituted aryl, —R⁶—OR⁷, —R⁶—CN and —R⁶—C(O)OR⁸;
each R⁴ and R⁵ is independently hydrogen, alkyl, or —OR⁷;
each R⁶ is independently a direct bond or a straight or branched alkylene chain;
each R⁷ is hydrogen, alkyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

32. The method of claim 31 wherein the compound of formula (If) is (3,4-dimethylthieno[2,3-b]thiophene-2,5-diyl)bis(methylene)dicarbamimidothioate.

33. The method of claim 1 wherein the compound of formula (I) is a compound of formula (Ig): 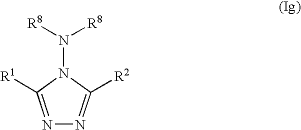
wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

34. The method of claim 33 wherein the compound of formula (I) is a compound of formula (Ig) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

35. The method of claim 34 wherein the compound of formula (I) is a compound of formula (Ig) wherein:
R¹ and R² are both —R⁶—S—C(═NR⁴)N(R⁴)R⁵;
each R⁴ and R⁵ is independently hydrogen, alkyl, or —OR⁷;
R⁶ is a direct bond or a straight or branched alkylene chain;
R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
each R⁸ is independently hydrogen or alkyl.

36. The method of claim 35 wherein the compound of formula (Ig) is (4-amino-4H-1,2,4-triazole-3,5-diyl)bis(methylene)dicarbamimidothioate.

37. The method of claim 1 wherein the compound of formula (I) is a compound of formula (Ih): 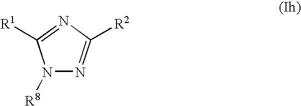
wherein:
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
R⁸ is independently hydrogen or alkyl.

38. The method of claim 37 wherein the compound of formula (I) is a compound of formula (Ih) wherein:
R¹ and R² are the same and selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵, —R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
R⁸ is independently hydrogen or alkyl.

39. The method of claim 38 wherein the compound of formula (I) is a compound of formula (Ih) wherein:
R¹ and R² are both —R⁶—S—C(═NR⁴)N(R⁴)R⁵;
each R⁴ and R⁵ is independently hydrogen, alkyl, or —OR⁷;
R⁶ is a direct bond or a straight or branched alkylene chain;
R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl; and
R⁸ is independently hydrogen or alkyl.

40. The method of claim 39 wherein the compound of formula (Ih) is (1H-1,2,4-triazole-3,5-diyl)bis(methylene)dicarbamimidothiodate.

41. A method of treating an iron disorder in a mammal by the inhibition of DMT1 in the mammal, wherein the method comprises administering to the mammal in need thereof a therapeutically effective amount of a compound of formula (I): 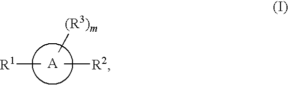
wherein:
m is 0, 1, 2, 3, or 4; 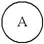
is aryl or heteroaryl;
R¹ and R² are each independently selected from the group consisting of —R⁶—S—C(═NR⁴)N(R⁴)R⁵, —R⁶—C(O)—S—C(═NR⁴)N(R⁴)R⁵, —R⁶—S—C(═NR⁴)N(R⁴)N(R⁴)R⁵, —R⁶—O—C(═NR⁴)N(R⁴)R⁵,
—R⁶—C(O)—N═C[N(R⁴)(R⁵)]N(R⁴)R⁵, —R⁶—C(═NR⁴)N(R⁴)R⁵, —R⁶—C(═NCN)N(R⁴)R⁵, —R⁶—N(R⁷)C(═NCN)N(R⁴)R⁵ and —R⁶—N(R⁷)C(═NR⁴)N(R⁴)R⁵;
each R³ is independently selected from the group consisting of hydrogen, alkyl, halo, haloalkyl, optionally substituted aryl, —R⁶—OR⁷, —R⁶—CN, —R⁶—NO₂, —R⁶—N(R⁸)₂, —R⁶—C(O)OR⁸, —R⁶—C(O)N(R⁸)₂, —N(R⁸)S(O)ₜR⁹, —S(O)ₜOR⁹, —S(O)ₚR⁸, —S(O)ₜN(R⁸)₂, —R⁶—S—C(═NR⁴)N(R⁴)R⁵, —R⁶—O—C(═NR⁴)N(R⁴)R⁵, —R⁶—C(═NR⁴)N(R⁴)R⁵, and —R⁶—N(R⁷)—C(═NR⁴)N(R⁴)R⁵, wherein each t is independently 1 or 2 and each p is 0, 1 or 2;
each R⁴ and R⁵ is independently hydrogen, alkyl, or —OR⁷;
each R⁶ is independently a direct bond or a straight or branched alkylene chain;
each R⁷ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R⁸ is independently hydrogen or alkyl; and
each R⁹ is alkyl;
as a stereoisomer, enantiomer, tautomer thereof or mixtures thereof;
or a pharmaceutically acceptable salt, solvate or prodrug thereof.
